(19) United States
(12) Patent Application Publication
(10) Pub. No.: US 2023/0248466 A1
(43) Pub. Date: Aug. 10, 2023

(54) LIGHTHEAD HAVING INTEGRATED AMBIENT ENVIRONMENTAL CONDITION SENSOR

(71) Applicant: American Sterilizer Company, Mentor, OH (US)

(72) Inventors: James A. Petrucci, Kirtland, OH (US); David A. Westenfelder, II, Mantua, OH (US); Richard P. Nardo, Highland Heights, OH (US); Joseph J. Groszek, Rocky River, OH (US)

(21) Appl. No.: 18/167,188

(22) Filed: Feb. 10, 2023

Related U.S. Application Data
(60) Provisional application No. 63/308,965, filed on Feb. 10, 2022.

(51) Int. Cl. A61B 90/35; H05B 47/105; F24F 11/89; G05B 19/042

(52) U.S. Cl. CPC A61B 90/35; H05B 47/105; F24F 11/89; G05B 19/042; F24F 2110/65

(57) ABSTRACT

A system includes a surgical lighthead including a lighthead housing including a plurality of light emitting elements therein that are arranged to emit light toward to a region of interest, and a handle attached to the lighthead housing. A sensor is integrated with the lighthead and configured to measure an ambient environmental condition at the lighthead. In other embodiments, a medical device support system includes a support and an ambient environmental condition sensor assembly coupled to the support column and including a sensor configured to measure an ambient environmental condition at the ambient environmental condition sensor assembly. A controller is operatively coupled to the sensor and configured to detect an ambient environmental condition based at least in part on the measured ambient environmental condition by the sensor.

### FIELD OF THE INVENTION

The technology of the present disclosure relates generally to a surgical lighting system, and more specifically to a lighthead for a medical device support system and/or suspension system including one or more ambient environmental condition sensors.

### BACKGROUND

The environmental conditions of rooms associated with health treatment such as hospital examination rooms, clinics, surgery rooms and emergency rooms are typically monitored and controlled. Exemplary environmental conditions include temperature, humidity, pressure, particulates, and air flow. Control of one or more of these environmental conditions are intended to assist in the safety of a patient during a medical procedure, as well as the comfort of healthcare professionals and/or patient.

However, it can be challenging to provide precise control that is appropriate for a medical procedure taking place, as environmental conditions can vary throughout the room during the medical procedure. Operating rooms typically have diffusers or air handling systems. Quick air turnover leads to varied air quality and varied ambient conditions at different points in the room. Also, in an operating room, equipment is typically concentrated around the surgical table. Common examples of equipment that can affect the environmental conditions are surgical lights, electro surgery tools, surgical energy devices, surgical tables, camera system, light source, bypass machine, patient warmer, insufflator, EKG, anesthesia cart, and the like. Healthcare professionals and staff also typically gather around the surgical table. This can create a localized area of varied temperature, humidity, air quality, and air flow in the vicinity of the patient and/or the healthcare professionals.

Conventional air handling systems include wall-mounted sensors to detect environmental conditions, which are insufficient for implementing effective control of these localized varied environmental conditions. For example, during an operation procedure, if ambient conditions near the patient are sufficiently different than ambient conditions at the wall, it is not ideal to control HVAC based on a thermostat mounted at the wall of the room. Surgical lights and equipment such as that listed above may create a local hot spot around the surgeon (and surgical site). If the surgeon requests the circulator to reduce the temperature in the room, the circulator checks the wall mounted thermostat sampling air temperature far from the surgical field and reports that the temperature is at the lower acceptable limit and will not adjust the HVAC system. The surgeon is in a local hot spot and performs the surgery in less-than-ideal temperature conditions. This can place stress on the surgeon and on the patient.

It is also difficult and obtrusive to place dedicated equipment in the vicinity of the surgical table for detecting environmental conditions, particularly in view of the crowded conditions around the surgical table and frequent need to adjust positioning of equipment and healthcare professionals and staff. Additionally, portions of the area in the vicinity of the surgical table is part of the sterile field which has different environmental characteristics than those of the nonsterile areas in the OR. And while handheld equipment can in some cases be used by healthcare professionals and staff to periodically detect environmental conditions, this handheld equipment is not integrated with the environmental control systems of the room, and is not used in a manner that allows for active monitoring and control of the environmental control system. For environmental systems that impact the sterile field it is imperative to have the sensing technology in this space without impacting the control.

Accordingly, there remains a need for further contributions in this area of technology.

### SUMMARY OF INVENTION

The present disclosure relates to a surgical lighthead having one or more ambient environmental condition sensors integrated therewith. Lightheads for medical device support systems, suspension systems and/or other carry systems, are used in health treatment settings such as hospital examination rooms, clinics, surgery rooms and emergency rooms to illuminate a region of interest (e.g., surgical treatment site or other medical site) below or proximate the lighthead. The lightheads typically include a housing, one or more light emitting elements mounted inside the housing, and a handle mounted to the housing to enable a healthcare professional or other individual to adjust the position of the lighthead according to the needs of a specific medical procedure. The one or more ambient environmental condition sensors may be integrated into the handle of the lighthead and/or into the housing of the lighthead.

By integrating the one or more ambient environmental condition sensors into the lighthead, improved control of the environmental conditions can be provided in the vicinity of the patent and/or healthcare professionals. Because the lighthead is typically used to illuminate the region of interest, the sensors integrated therein are also accordingly adjusted and placed in the vicinity of the surgical table. Integration of the one or more environmental condition sensors into the handle of the lighthead also minimizes or eliminates any obstruction provided by sensing equipment that would otherwise be implemented in the vicinity of the surgical table.

Other embodiments of the present disclosure related to medical device support system including a dedicated suspension arm that supports one or more ambient environmental condition sensors. Improved control of the environmental conditions can be provided in the vicinity of the patent and/or healthcare professionals by positioning the suspension arm in a manner in which the one or more ambient environmental condition sensors are adjusted and placed in the vicinity of the surgical table. Integration of the one or more ambient environmental condition sensors also minimizes or eliminates any obstruction provided by sensing equipment that would otherwise be implemented in the vicinity of the surgical table.

In accordance with one aspect of the present application, a system includes: a surgical lighthead including a lighthead housing including a plurality of light emitting elements therein that are arranged to emit light toward to a region of interest, and a handle attached to the lighthead housing; a sensor integrated with the lighthead and configured to measure an ambient environmental condition at the lighthead; and a controller operatively coupled to the sensor and configured to detect an ambient environmental condition based at least in part on the measured ambient environmental condition by the sensor.

In some embodiments, the sensor is a thermistor and the ambient environmental condition is temperature.

In some embodiments, the sensor is a thermocouple and the ambient environmental condition is temperature.

In some embodiments, the sensor is a thermometer and the ambient environmental condition is temperature.

In some embodiments, the sensor is an infrared temperature sensor and the ambient environmental condition is temperature.

In some embodiments, the sensor is a hygrometer and the ambient environmental condition is humidity.

In some embodiments, the sensor is a barometer and the ambient environmental condition is atmospheric pressure.

In some embodiments, the lighthead includes one or more baffles configured to direct airflow to the sensor.

In some embodiments, the sensor is a particle counter and the ambient environmental condition is concentration of particles in the air.

In some embodiments, the sensor is a reactive oxygen species sensor and the ambient environmental condition is concentration of reactive oxygen species in the air.

In some embodiments, the sensor is located in a flow path in the handle, an air inlet of the flow path is in fluid communication with the sensor, and an air outlet of the flow path is in fluid communication with the sensor.

In some embodiments, the handle includes an air filter.

In some embodiments, the sensor is integrated with the handle of the lighthead.

In some embodiments, the sensor is housed in the housing of the lighthead.

In some embodiments, the controller is configured to control the ambient environmental condition based at least in part on the measured ambient environmental condition by the sensor.

In some embodiments, the controller is configured to control an HVAC system based at least in part on the ambient environmental condition measured by the sensor.

In some embodiments, the controller is configured to control an air purification system based at least in part on the ambient environmental condition measured by the sensor.

In some embodiments, the controller is configured to control a heated or cooled blanket based at least in part on the ambient environmental condition measured by the sensor.

In some embodiments, the controller is configured to control a heated or cooled underbody pad based at least in part on the ambient environmental condition measured by the sensor.

In some embodiments, the controller is configured to control a heated or cooled headrest based at least in part on the ambient environmental condition measured by the sensor.

In some embodiments, the controller is configured to control light output intensity of the plurality of the light emitting elements based at least in part on the ambient environmental condition measured by the sensor.

In accordance with another aspect of the present application, a medical device support system includes: a support; and the system of any one of the above-referenced embodiments, wherein the surgical lighthead is mounted to the medical device suspension system.

In accordance with another aspect of the present application, a medical device support system includes: a support; an ambient environmental condition sensor assembly coupled to the support column and including a sensor configured to measure an ambient environmental condition at the ambient environmental condition sensor assembly; and a controller operatively coupled to the sensor and configured to detect an ambient environmental condition based at least in part on the measured ambient environmental condition by the sensor.

In some embodiments, the medical device support system further includes a lighthead coupled to the support column.

In some embodiments, the sensor is a thermistor and the ambient environmental condition is temperature.

In some embodiments, the sensor is an infrared temperature sensor and the ambient environmental condition is temperature.

In some embodiments, the sensor is a thermocouple and the ambient environmental condition is temperature.

In some embodiments, the sensor is a thermometer and the ambient environmental condition is temperature.

In some embodiments, the sensor is a hygrometer and the ambient environmental condition is humidity.

In some embodiments, the sensor is a barometer and the ambient environmental condition is atmospheric pressure.

In some embodiments, the sensor is a particle counter and the ambient environmental condition is concentration of particles in the air.

In some embodiments, the sensor is a reactive oxygen species sensor and the ambient environmental condition is concentration of reactive oxygen species in the air.

These and further features will be apparent with reference to the following description and attached drawings which set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings. The invention includes all changes, modifications and equivalents coming within the spirit and terms of the claims appended hereto.

Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

The annexed drawings, which are not necessarily to scale, show various aspects of the present disclosure.

FIG. 1 is a side elevation view of an overall configuration of a medical device support system in accordance with an embodiment of the present disclosure, showing a top of a left positioned lighthead and a bottom of a right positioned lighthead.

FIG. 2 is a side cross section view of a lighthead in accordance with an embodiment of the present disclosure, showing a housing base, a housing cover, and internal components of the lighthead and handle.

FIG. 3 is a perspective side view of a handle in accordance with an embodiment of the present disclosure.

FIG. 4 is a bottom view of the handle of FIG. 3.

FIG. 5 is a schematic view of an exemplary flow path including ambient environmental condition sensors.

FIG. 6 is a schematic side view of an exemplary room including a medical device support system.

FIG. 7 is a perspective view of a lighthead in accordance with an embodiment of the present disclosure.

FIG. 8 is a side cross section view of a lighthead in accordance with an embodiment of the present disclosure, showing a housing base, a housing cover, and internal components of the lighthead and handle.

FIG. 9 is a side elevation view of an overall configuration of a medical device support system in accordance with an embodiment of the present disclosure, showing a top of a left positioned lighthead and a suspension arm including ambient environmental condition sensors.

FIG. 10 is a schematic side view of an exemplary medical device support system arranged relative to an operating table.

FIG. 11 is a schematic block diagram of an exemplary control system.

FIGS. 12 and 13 are schematic block diagrams of exemplary environmental condition control systems.

FIGS. 14-17 are schematic block diagrams of exemplary control systems.

FIG. 18 is a schematic block diagram of an exemplary environmental condition control system

FIG. 19 is a flow chart showing an exemplary process for determining a measured ambient environmental condition proximate a region of interest.

FIGS. 20-26 are flowcharts showing exemplary process for controlling an environmental condition proximate the region of interest based at least in part on the environmental condition measured by the ambient environmental condition sensor.

### DETAILED DESCRIPTION

For the purpose of promoting an understanding of the principles of the present disclosure, reference will now be made to the embodiments illustrated in the drawings and specific language will be used to describe the same. It will nevertheless be understood that no limitation of the scope of the present disclosure is thereby intended. The figures are not necessarily to scale. Features that are described and/or illustrated with respect to one embodiment may be used in the same way or in a similar way in one or more other embodiments and/or in combination with or instead of the features of the other embodiments. Any alterations and further modifications of the described embodiments, and any further applications of the principles of the present disclosure as described herein, are contemplated as would normally occur to one skilled in the art to which the present disclosure relates.

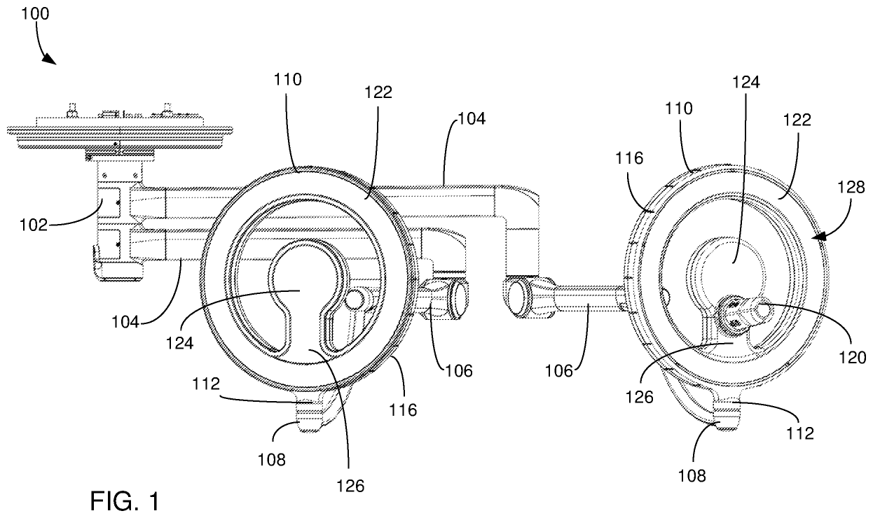

With reference to FIG. 1, an exemplary medical device support system is shown at 100. The medical device support system 100 includes a central shaft or support column 102 that is suspended from the ceiling, and two generally horizontal extension arms 104 mounted to the shaft 102 for rotational movement about the central shaft 102. In other implementations, the central shaft 102 could be mounted to a wall or stand rather than the ceiling. Two load balancing arms 106 are pivotably mounted to the distal ends of the respective extension arms 104. Yoke assemblies 108 are mounted to the distal ends of the respective load balancing arms 106. The yoke assemblies 108, in turn, support respective lightheads 110 for multi-axis movement relative to the load balancing arms 106. Each lighthead 110 includes a bushing or other coupling member 112 that rotatably connects the lighthead 110 to the distal end of an arm of a respective yoke assembly 108, as shown. The load balancing arms 106 and yoke assemblies 108 enable positioning of the lightheads 110 to a desired orientation relative to, for example, a patient operating table and healthcare professionals in the operating room.

The exemplary medical device support system shown in FIG. 1 includes two lightheads 110, each mounted to a respective extension arm 104, load balancing arm 106, and yoke assembly 108. It will be appreciated that in other embodiments, the medical device support system may include more or fewer lightheads. It will also be appreciated that the medical device support system may include other accessories mounted to the central shaft 102.

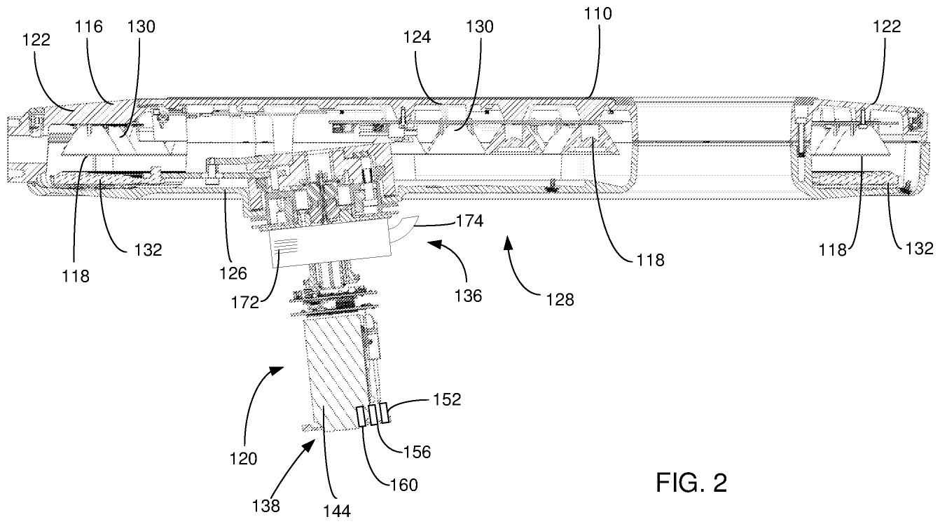

With additional reference to FIG. 2, the lighthead 110 includes a housing 116, one or more light emitting elements 118 mounted inside the housing, and a handle 120 mounted to the housing to enable a healthcare professional or other individual to adjust the position of the lighthead according to the needs of a specific medical procedure. In the illustrated example, the lighthead 110 includes an annular shape outer portion 122, an inner round portion 124 that is concentric with the outer portion, and a radially protruding arm 126 that connects the annular shape outer portion 122 to the inner round portion 124. The handle is connected to the radially protruding arm. This configuration of the lighthead 110 allows for air flow to flow through the gap between the concentric outer portion 122 and inner portion 124, resulting in more effective contact and interaction of the air with one or more sensors in the handle.

As shown in FIG. 2, the housing 116 supports the plurality of light emitting elements 118. The light emitting elements 118 may in some embodiments include one or more solid-state light emitters. Exemplary solid-state light emitters include such devices as light emitting diodes (LEDs), laser diodes, and organic LEDs (OLEDs). The LEDs may be broad spectrum LEDs (e.g., white light emitters) or LEDs that emit light of a desired color or spectrum (e.g., red light, green light, blue light, or ultraviolet light). In other embodiments, the LEDs may be a mixture of broad-spectrum LEDs and LEDs that emit narrow-band light of a desired color, or a mixture of LEDs that emit light of different respective colors or spectrum. In some embodiments, the solid-state light emitters constituting the light emitting elements 118 all generate light having the same nominal spectrum. In other embodiments, at least some of the solid-state light emitters constituting the light emitting elements 118 generate light that differs in spectrum from the light generated by the remaining solid-state light emitters. In other embodiments, the light emitting elements 118 may include one or more other types of light sources. Non-limiting examples of other types of light sources include incandescent and gas discharge light sources. In still other embodiments, the light emitting elements 118 may include a combination of solid-state light emitters and one or more of the above other types of light sources.

Light from the light emitting elements is emitted from a light emitting face 128 (i.e., light emitting side) of the lighthead. The lighthead may include one or more elements configured to affect the light output distribution of the light from the lighthead. With continued reference to FIG. 2, a plurality of collimators 130 are mounted to the inside surface of the housing 116 and in the light emitting paths of the respective plurality of light emitting elements 118. Each collimator may be associated with a respective light emitting element 118 and may be arranged to collect and direct, and/or collimate, the light emitted from the associated light emitting element 118 into a narrowed beam. In the illustrative embodiment, the housing 116 also includes a lens 132, that is shaped to redirect light emitted from the light emitting elements and passing therethrough. The lens 132 can take on any form for spreading and/or bending the light emitted by the light emitting elements 118. The lens may be adjusted to adjust the spreading, focusing, and/or bending the light. In other embodiments, the lens and/or the collimators may be omitted from the lighthead.

A controller (402, 602) controls the light emitting elements 118 to emit light to a region of interest (e.g., surgical treatment site or other medical site) below or proximate the lighthead 110. For example, a controller may control the light emitting elements 118 of the annular shape outer portion 122 and the inner portion 124 to emit light to a region of interest below the lighthead 100. Control of the respective light emitting elements 118 may be performed, for example, collectively, individually, in groups, by section, or in any other suitable manner. In some embodiments, the controller may be provided as part of the lighthead 110. In other embodiments, the controller may be implemented elsewhere in the medical device support system 100, for example external to the lighthead 110, or the controller may be implemented external to the medical device support system 100.

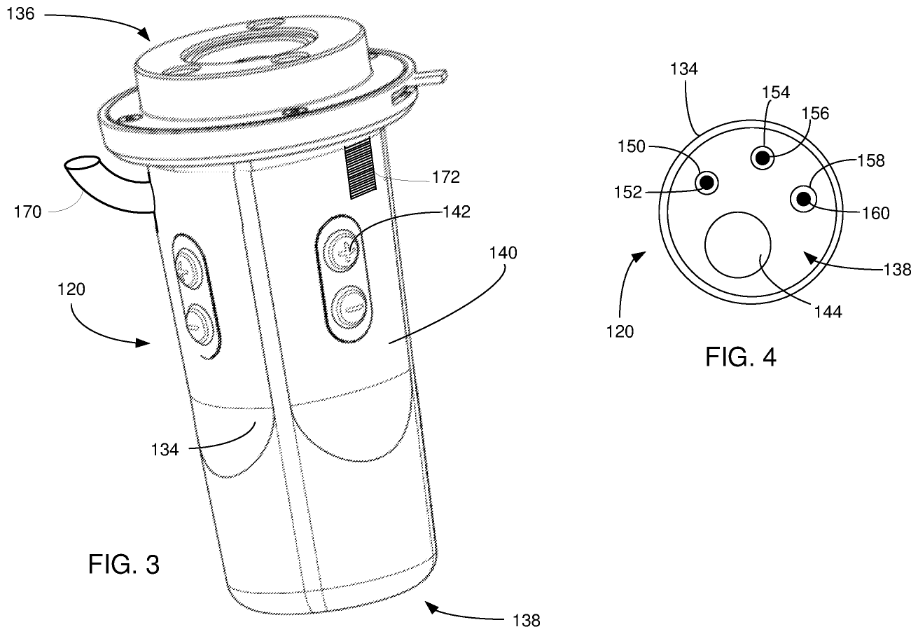

The handle 120 is attached to the lighthead housing and extends between a proximate end 136 proximate the housing and a distal end 138 distal the housing. With additional reference to FIG. 3, the handle 120 includes a handle housing 134 that has generally tubular shape extending between the proximate end 136 and distal end 138. The tubular shape may be cylindrical in shape, as shown, or non-cylindrical in shape. The handle 120 includes a grip portion 140 including one or more buttons 142. In some embodiments, the one or more buttons provide a user interface for controlling one or more attributes of the emitted light from the lighthead 110. For example, the one or more buttons may be used as an input for a user to adjust the color temperature, intensity, and/or distribution of the light. The handle housing 134, including the grip portion 140 thereof, has a sufficient size to be gripped by a human hand meaning that the outermost diameter or perimeter of the handle housing 134 is selected to enable a human hand to be wrapped around the handle housing. In the embodiment shown, a camera 144 is housed in the handle 120. The camera may be arranged in the handle to capture images of a region of interest. For example, the camera 144 may be arranged and oriented so that its lens can capture images through a port at the distal end 138 of the handle 120.

It will be appreciated that the specific configuration of the lighthead 110, including the shape of the housing, the arrangement of the light emitting elements 118, and the location and configuration of the handle 120 can be provided in any suitable configuration. For example, an annular shape outer portion 122 and the inner round portion 124 need not be in concentric relation to one another and instead can be arranged by the protruding arm in eccentric relation to one another. In another example, the inner round portion 124 of the lighthead 110 may be omitted; and in such form, only the annular shape outer portion 122 emits light to the region of interest (e.g., surgical treatment site or other medical site) below or proximate the lighthead. In another example, the housing may be configured as a different shape (e.g., rectangle, square, circle, hexagon, octagon, etc.) and the light emitting elements can be accordingly arranged in the housing. In another example, the handle can be provided at a different location on the light emitting side of the housing, on a perimeter of the housing, or on the side of the housing opposite the light emitting side.

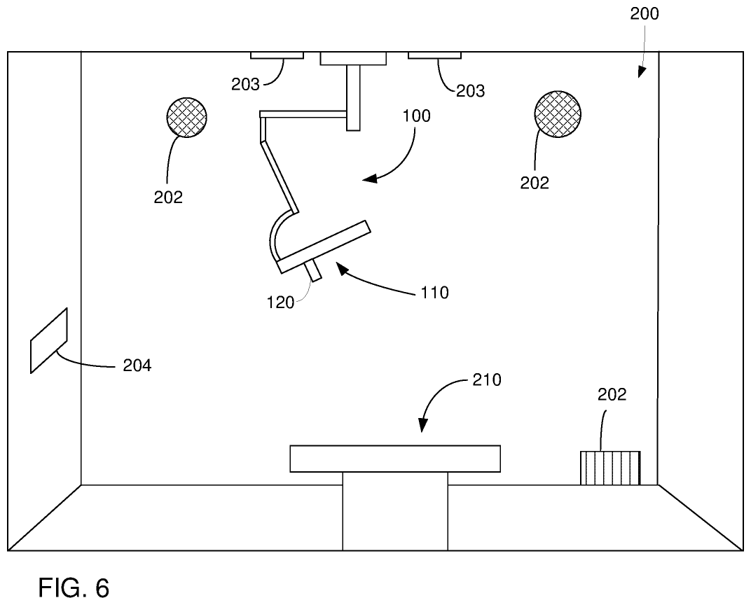

With additional reference to FIG. 6, the medical device support system 100 may be installed in a room 200 to provide light to a specific area or region of the room. The room may be, for example, an operating room, and the region of interest may be, for example, an operating table, surgical treatment site, or other medical site. In the embodiment shown, the medical device support system 100 is fixed to the ceiling.

One or more environmental temperature control systems may be implemented in the room 200. For example, an HVAC system may provide heating and/or cooling in the room. The HVAC system may include components such as a blower motor, filter, fan, etc. One or more air ducts, vents, registers, and/or returns 202 included as part of the HVAC system may be present in the room at predetermined locations to effect heating and/or cooling. An air purification system may provide purified air proximate the region of interest. The air purification system may include components such as a blower motor, filter, fan, etc. One or more air ducts, vents, registers, and/or returns 203 included as part of the HVAC system may be present in the room at predetermined locations. The air purification system may be configured to provide laminar flow of air around the patient or region of interest 210. The laminar flow air may encircle the region of interest 210 and assist in prevention of contamination. In some embodiments, the air purification system is integrated together with a part of the HVAC system. In other embodiments, the air purification system is separate from the HVAC system.

One or more user inputs 204 such as a control panel may be included that allow for a user to adjust the environmental conditions in the room (e.g., room temperature). The one or more user inputs 204 may be embodied, for example, as a touch panel that can display a user interface, one or more hard buttons or knobs, and the like. The one or more user inputs can be used to control, for example, the HVAC system, the air purification system, and/or the lighthead.

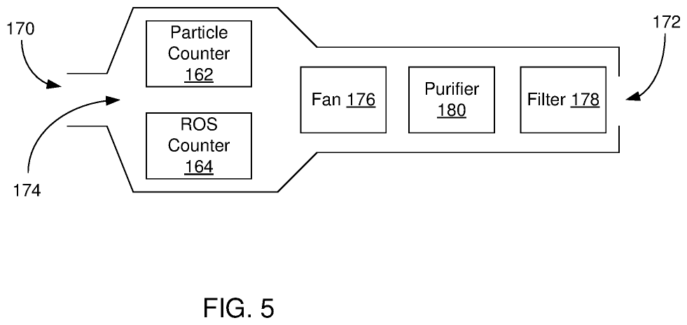

With continued reference to FIGS. 1-5, in some embodiments, the lighthead includes one or more ambient environmental condition sensors. The one or more ambient environmental condition sensors measure one or more ambient environmental conditions proximate the lighthead. By positioning the lighthead proximate the region of interest 210 (e.g., for purposes of directing light toward the region of interest), the ambient environmental conditions measured by the one or more ambient environmental condition sensors allows for more accurate monitoring of the ambient environmental condition(s) proximate the region of interest 210. This may allow for more precise control of the environmental condition(s) in an area of the room in which a patient and/or healthcare professionals may be located. The one or more ambient environmental condition sensors may be coupled to the controller (402, 602). The controller may provide power to the one or more environmental sensors and may control the operation thereof.

FIGS. 3-5 show an exemplary embodiment of the handle 120. The handle 120 includes one or more ambient environmental condition sensors integrated therein.

In some embodiments, the handle includes one or more temperature sensors as an ambient environmental condition sensor to measure temperature. Room temperature can impact patient temperature, which can impact health outcomes. Room temperature also has a large effect on the comfort of healthcare professionals in the room. Placing the temperature sensor near the sterile field gives a more realistic snapshot of operating room ambient temperature especially as it relates to the patient or operating room staff. In an example, the temperature sensor is a thermistor. In another example, the temperature sensor is a thermocouple. In another example, the temperature sensor is a thermometer. In another example the temperature sensor is an infrared temperature sensor. In some examples, the infrared temperature sensor is an infrared thermometer that reacts to infrared radiation emitted by the object being measured. A lens may focus the infrared thermal radiation from the object on the sensor. The sensor converts the radiant power to an electrical signal. In other examples, the temperature sensor is embodied as an infrared camera that creates an image of the spatial distribution of infrared radiation. Images and/or video of a field of view can be captured by the camera, which may include a target in a region of interest illuminated by the one or more light emitting elements. In other examples, the handle includes a combination of two or more different temperature sensors (e.g., a thermocouple and an infrared temperature sensor).

In the exemplary embodiment shown, the distal end of the handle housing 138 includes a port 150 through which the temperature sensor 152 is exposed to the ambient atmospheric conditions. It will be appreciated that in embodiment in which more than one temperature sensor is included, there may be more than one port in the handle housing through which the temperature sensors are respectively exposed to the ambient atmospheric conditions.

The signal from the temperature sensor representative of the measurement can be input to the controller to determine the ambient temperature proximate the lighthead and region of interest. The measured temperature can be used in the monitoring and control of the temperature around the region of interest (e.g., around the healthcare professionals, staff, and patient). As an example, the measured temperature can be used by the HVAC system and/or air purification system to control the temperature proximate the lighthead and region of interest. Exemplary control may maintain the temperature between 65° F. and 75° F.

In some embodiments, the handle includes a humidity sensor as an ambient environmental condition sensor to measure relative humidity. Humidity can impact the efficacy of electrosurgical units. In an example, the humidity sensor is a hygrometer. In the exemplary embodiment shown, the distal end of the handle housing includes a port 154 through which the humidity sensor 156 is exposed to the ambient atmospheric conditions.

The signal from the humidity sensor representative of the measurement can be input to the controller to determine the ambient relative humidity proximate the lighthead and region of interest. The measured relative humidity can be used in the monitoring and control of the relative humidity around the region of interest (e.g., around the healthcare professionals, staff, and patient). As an example, the measured relative humidity can be used with the HVAC system and/or air purification system to control the relative humidity proximate the lighthead and region of interest. Exemplary control may maintain the relative humidity between 20% and 60% Rh.

In some embodiments, the handle includes a pressure sensor to measure atmospheric pressure. It is often desired to maintain a positive pressure in an operating room. The measured atmospheric pressure can be used to monitor and change if needed the atmospheric pressure. The measured atmospheric pressure could be used, for example, to dose oxygen or adjust ventilator settings. Pressure monitoring can also be used to monitor how often and for how long operating room doors are opened during a medical procedure. Door openings are a metric under investigation for correlation with hospital acquired infections. Monitoring door openings may be helpful in a hospital initiative to minimize doors opening. In an example, the pressure sensor is a barometer. The barometer may be a separate sensor from the humidity sensor (e.g., hygrometer). Alternatively, the barometer may be integrated with the humidity sensor (e.g., a hygrometer barometer). In the exemplary embodiment shown, the distal end of the handle housing 138 includes a port 158 through which the barometer 160 is exposed to the ambient atmospheric conditions.

The signal from the pressure sensor representative of the measurement can be input to the controller to determine the ambient atmospheric pressure proximate the lighthead and region of interest. The measured atmospheric pressure can be used in the monitoring and control of the relative humidity around the region of interest (e.g., around the healthcare professionals, staff, and patient).

It will be appreciated that while the location of the ports 150, 154, 158 and sensors 152, 156, 160 are shown as being located at the distal end of the handle, in other embodiments they may be located in any suitable location on the handle. It will also be appreciated that in some embodiments, the port(s) associated with the sensors may be respectively provided in different configurations or may be omitted. In some embodiments, a cover may be placed over the handle, and may cover one or more of the ports and sensors.

In some embodiments, the handle includes one or more ambient environmental condition sensors to detect the concentration of one or more components in the air. Air purification systems, where purified air is directed at the patient, may include air monitoring. Air quality sensors should be close to the surgical site to offer information that impacts the patient. For example, in some embodiments, the ambient environmental condition sensor is a particle counter 162 to measure the concentration of particles in the air. The ambient environmental condition sensors may in some embodiments be used to monitor the health of the HVAC or air purification system and could provide feedback or early warning of the need for service to the system. In another example, the environmental sensor is a reactive oxygen species (ROS) sensor 164 to measure the concentration of reactive oxygen species in the air. For air purification technologies that expel a reactive oxygen species to destroy pathogens, it is desirable to measure the amount of reactive oxygen species created and control the concentration. The measured concentration is most relevant close to the surgical site. The ambient environmental condition sensors may in some embodiments be used in providing feedback to increase or decrease ROS production.

The one or more ambient environmental condition sensors may be disposed within the handle housing and in fluid communication with the atmosphere. Ambient air may be directed through a flow path through the handle to be brought into contact with the one or more sensors. As shown in FIGS. 2, 3, and 5, an inlet 170 extends through the housing 134 to draw air and is in fluid communication with the one or more ambient environmental condition sensors. An outlet 172 extends through the housing 134 to expel the input air and is in fluid communication with the one or more sensors 162, 164. A flow path 174 is provided between the inlet and the outlet, and the one or more ambient environmental condition sensors 162, 164 are located in the flow path 174. The inlet 170 and outlet 172 are arranged such that they are unobstructed by any cover that may be used with the handle. The inlet 170 and the outlet 172 may also be arranged such that recycling effect of the measured air is avoided. In the example shown, the inlet and outlet are located at circumferentially opposite sides of the handle. A fan 176 is provided in the flow path to effect movement of air into the inlet 172, through the flow path 174, and out the outlet 172. In some embodiments, a filter 178 is in the flow path 174 to filter the sampled air prior to being output from the outlet 172. In some embodiments, an air purifier 180 (e.g., UV light air purifier) is in the flow path 174 to purity the sampled air prior to being output from the outlet 172. The filter 178 and/or air purifier 180 may maintain the integrity of the sterile field within which the sensor is located.

In the exemplary embodiment shown, the inlet 170 is embodied as a tube and the outlet 172 is embodied as a vent. In other embodiments, the inlet and outlet may have other suitable configurations. For example, the inlet may be embodied as a vent. In another example, the outlet may be embodied as a tube. In another example the airflow may be directed by tubes, baffles or other airflow directors to the sensors.

It will be appreciated that while the location of the sensors 162, 164 and the flow path 174 including the input 170 and outlet 172 are shown as being located at the proximal end of the handle, in other embodiments they may be located in any suitable location on the handle.

With reference to FIG. 6, the lighthead may be arranged such that it is a predetermined distance from the region of interest 210. Adjustment of the lighthead relative to the region of interest may be performed using the extension arm 104, load balancing arm 106, and/or yoke assembly 108. In an example, the lighthead may be adjusted such that it is a distance of about one meter from the region of interest. The one or more environmental conditions measured by the one or more ambient environmental condition sensors may provide a more accurate measurement of the environmental conditions proximate the lighthead and region of interest. This may provide improved control of the environmental conditions in the vicinity of the patent and/or healthcare professionals. The environmental conditions measured by the one or more sensors may be used by the healthcare professionals, HVAC system, air purification system, maintenance staff, regulatory organization, and/or other integrated system. In the embodiment in which the one or more ambient environmental condition sensors are used together with one or more of the above-mentioned systems, they may be integrated with the one or more systems using Operating Room Integration (ORI), Building Automation and Control network (BACnet), or any other communication protocol.

For example, the measured temperature and humidity can be used as an input to control the HVAC system to heat, cool, humidify, and/or dehumidify the room. As another example, the measured barometric pressure can be used as an input to the HVAC system to control the positive pressure condition of the room, as well as to detect a scenario in which the desired positive pressure of an operating room is lost. As another example, the measured particle count can be used as an input by the HVAC system or air purification system to indicate that the filtration system (e.g., filter) needs servicing. As another example, the measured ROS can be used as an input to the HVAC system or air purification system to adjust the production rate of ROS. As another example, the environmental conditions measured by the one or more ambient environmental condition sensors during a procedure may be recorded and stored as a record in connection with the procedure.

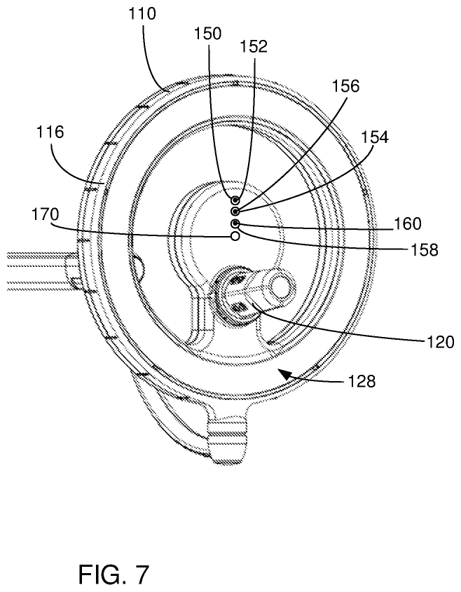

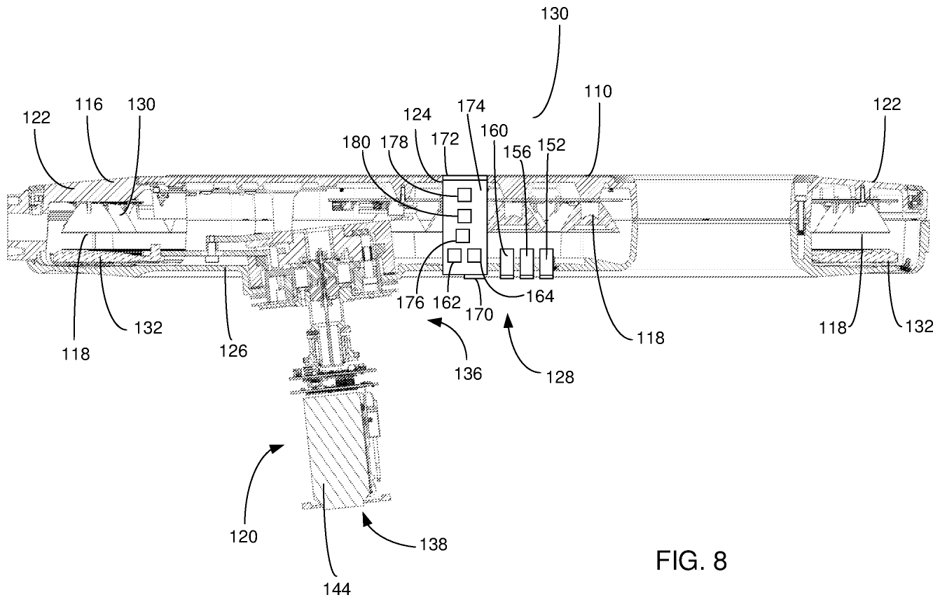

While the above-described embodiments include the one or more environmental sensors housed within the handle of the lighthead, in other embodiments, the one or more environmental sensors are included at a different location of the lighthead. For example, the one or more environmental sensors may be attached to and/or disposed in the housing 116. For example, FIGS. 7 and 8 show an embodiment in which the one or more environmental sensors are mounted to the housing at the inner round portion 124 of the lighthead. In other examples, the one or more environmental sensors are mounted to the housing at the annular shape outer portion 122 or the radially protruding arm 126. In embodiments in which there are multiple environmental sensors, the lighthead may include one or more ambient environmental condition sensors disposed in the housing and one or more ambient environmental condition sensors in the handle.

The one or more ambient environmental condition sensors may be any one of the ambient environmental condition sensors described above with respect to the embodiments of FIGS. 1-6. The details thereof will not be repeated for sake of brevity.

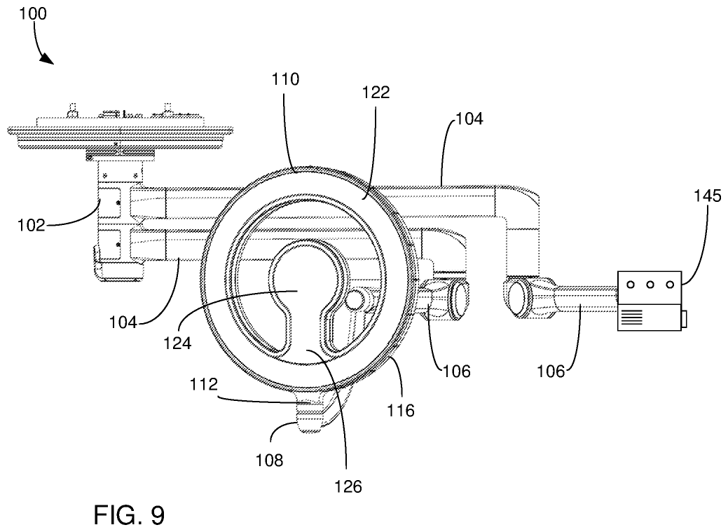

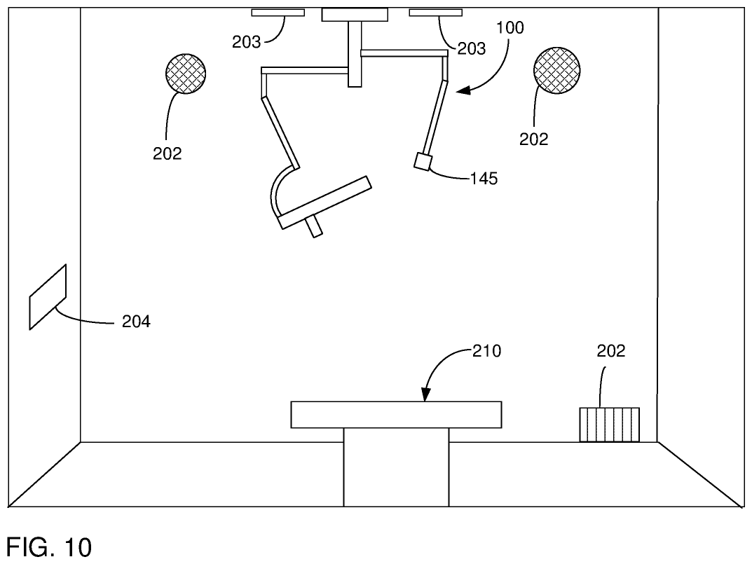

It will be appreciated that in other embodiments, the one or more ambient environmental condition sensors can be located in a different location, external to the lighthead. For example, FIGS. 9 and 10 show an example in which the medical device support system includes a designated suspension arm including an ambient environmental condition sensor assembly 145 including one or more ambient environmental condition sensors. The location of the ambient environmental condition sensor assembly may be adjusted via manipulation of the suspension arm so that the one or more ambient environmental condition sensors are arranged to detect the environmental conditions near the lighthead and the region of interest 210. The one or more ambient environmental condition sensors may be any one of the ambient environmental condition sensors described above with respect to the embodiments of FIGS. 1-6. The details thereof will not be repeated for sake of brevity.

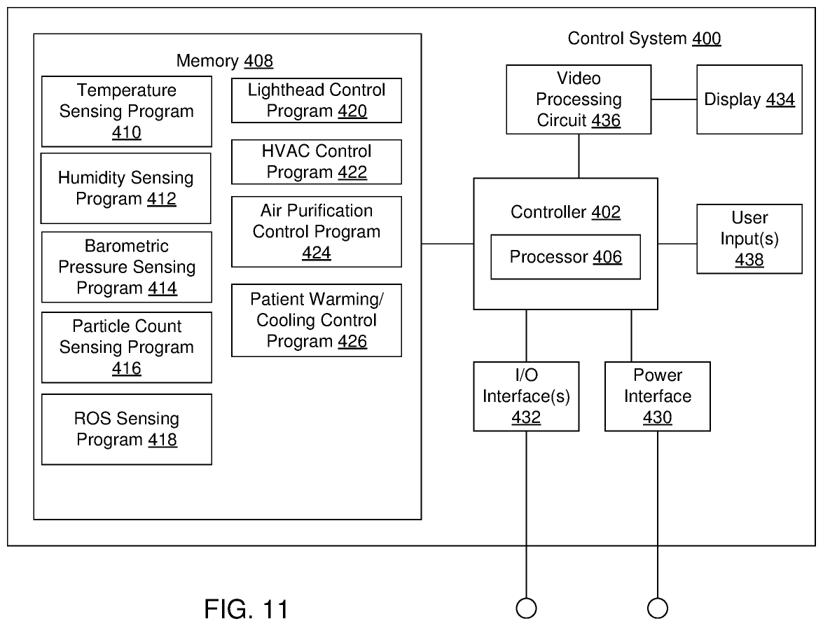

Referring now to FIG. 11, the one or more ambient environmental condition sensors are connected to a controller 402. The one or more ambient environmental condition sensors may be powered by and communicate with the controller. The controller may be implemented as part of a control system 400 and may be configured to process an input signal from the one or more ambient environmental condition sensors. The controller may control or may provide an output for controlling an environmental condition based at least in part on the input signal from the one or more ambient environmental condition sensors. Exemplary control can include one or more of the control of an HVAC system, air purification system, patient warming/cooling system, lighthead, and/or other system. The controller may be integrated with the one or more systems using ORI and/or BACnet communication protocol.

In some embodiments, the control system 400 is provided in the handle or in the lighthead. In other embodiments, the control system 400 is located external to the handle and lighthead, or outside of the medical device support system 100. In other embodiments, the control system 400 is located in a combination of two or more of the handle 120, the lighthead housing 116, outside of the lighthead housing 116, and outside of the medical device support system 100.

The controller 402 is configured to carry out overall control of the functions and operations of the control system 400. The controller 402 may include a processor 406, such as a central processing unit (CPU), microcontroller, or microprocessor. The processor 406 executes code stored in a memory (not shown) within the controller 402 and/or in a separate memory, such as the memory 408, in order to carry out operation of the lighthead and one or more ambient environmental condition sensors. For example, the memory may contain stored data pertaining to the operation of the one or more ambient environmental condition sensors, and the processing of the signal received from the one or more ambient environmental condition sensors. The memory may contain stored data pertaining to the control of the HVAC system, the control of the air purification system, the control of the lighthead, and/or the control of one or more devices of an active warming/cooling system.

FIG. 11 shows an example in which a temperature sensing program 410, humidity sensing program 412, barometric pressure sensing program 414, particle count sensing program 416, ROS sensing program 418, lighthead control program 420, HVAC control program 422, air purification control program 424, and patient warming/cooling control program 426 are stored in the memory 408. Each of these programs may be embodied in the form of executable logic routines (e.g., lines of code, software programs, etc.) that are stored on a non-transitory computer readable medium (e.g., the memory 408) and executed by the controller 402 (e.g., using the processor 406).

The memory 408 may be, for example, one or more of a buffer, a flash memory, a hard drive, a removable media, a volatile memory, a non-volatile memory, a random access memory (RAM), or other suitable device. In a typical arrangement, the memory 408 may include a non-volatile memory for long term data storage and a volatile memory that functions as system memory for the controller 402. The memory 408 may exchange data with the controller 402 over a data bus. Accompanying control lines and an address bus between the memory 408 and the controller 402 also may be present. The memory 408 is considered a non-transitory computer readable medium.

Operating power may be received from an external power source via a power interface 430.

The control system 400 may further include one or more input/output (I/O) interface(s) 432. The I/O interface(s) 432 may be in the form of one or more electrical connectors and may connect the controller 402 to the one or more ambient environmental condition sensors. For example, the controller may receive and process a signal from the temperature sensor 152, humidity sensor 156, barometric pressure sensor 158, particle counter 162, and/or ROS sensor 164. The I/O interface(s) 432 may connect the controller to one or more components of the HVAC system, air purification system, patient warming/cooling system, and/or lighthead.

The control system 400 may include a display 434. In some embodiments, the display 434 can display information such as the measured environmental conditions, set points, warnings, etc. The display 434 may be a lighted display. In some embodiments, the display 434 is a backlit liquid-crystal display (LCD). In other embodiments, the display 434 is an organic light-emitting diode (OLED) display. One exemplary embodiment of the display is the control panel 204 (FIGS. 6 and 10). The display 434 may be coupled to the controller 402 by a video processing circuit 436 that converts image and/or video data to an image and/or video signal used to drive the display 434. The video processing circuit 436 may include any appropriate buffers, decoders, video data processors and so forth.

The control system 400 may include one or more user inputs 438 for receiving user input for controlling operation of the control system 400. Exemplary user inputs 438 include, but are not limited to, a touch input that overlays the display 434 for touch screen functionality, one or more buttons such as those included on the handle or in a different location, and so forth. One exemplary embodiment of a user input is the control panel 204 (FIGS. 6 and 10).

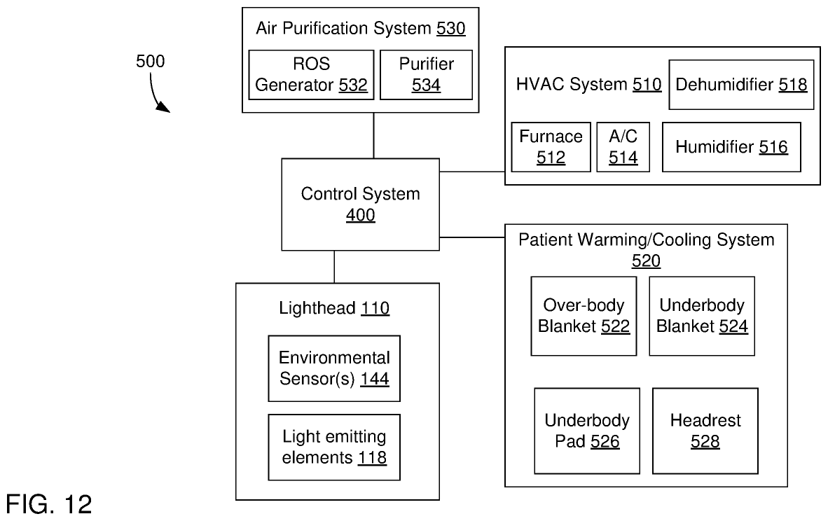

FIG. 12 shows an exemplary arrangement of an environmental condition control system 500. In the example shown, the control system 400 is coupled to the lighthead 110, HVAC system 510, patient warming/cooling system 520, and air purification system 530. While the control system 400 is schematically shown as being a separate unit from the lighthead 110, HVAC system 510, patient warming/cooling system 520, and air purification system 530, in other embodiments the control system 400 may be integrated within one or a combination of these systems. As described above, in some embodiments, the air purification system may be included as part of HVAC system.

The controller 402 of the control system 400 may be communicatively coupled to components of the lighthead 110. In some embodiments, the controller 402 is communicatively coupled to the light emitting elements 118. In other embodiments, the controller 402 is communicatively coupled to the one or more motors configured to adjust the lens of the lighthead. In some embodiments, this control can be accomplished by executing the lighthead control program 420 stored in the memory 408 for controlling overall operation of the lighthead 100. In an example, the controller controls the radiant power of the light emitting elements 118. In another example, the controller controls the position of the lens to adjust the light output distribution of the light emitting elements.

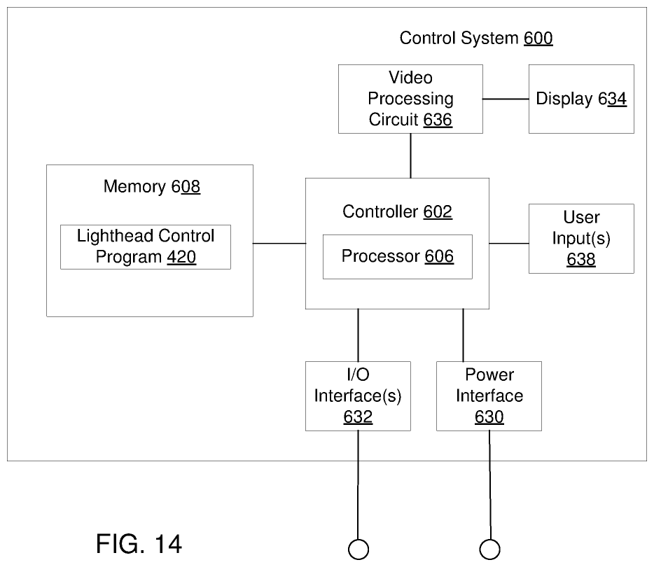

Alternatively, the controller 402 may be communicatively coupled to a separate controller for controlling the components of the lighthead. The separate controller may be configured to carry out overall control of the components of the lighthead, and the separate controller may include a processor (e.g., CPU) for executing code stored in a memory (see e.g., FIG. 14, controller 602). The components of the lighthead may be controlled by the separate controller based at least in part on values/signals received from controller 402. The controllers may communicate via ORI and/or BACnet communication protocol.

In some embodiments, the controller 402 may be communicatively coupled to the heating, ventilation and air-conditioning (HVAC) system. The controller 402 may control the operation of the HVAC system based at least in part on the signal of the one or more ambient environmental condition sensors. This control can be accomplished by the controller 402 executing the HVAC control program 422 stored in the memory 408 for controlling overall operation of the HVAC system 510. In an example, the controller controls the activation, deactivation, and/or setting of one or more of the blower, heater, compressor, condenser, and vents respectively associated with the furnace 512 and A/C 514 systems of the HVAC system based on the measured ambient temperature relative to the temperature setpoint. In another example, the controller controls the activation, deactivation, and/or setting of a dehumidifier 518 or humidifier 516 based on the measured relative humidity relative to the set relative humidity.

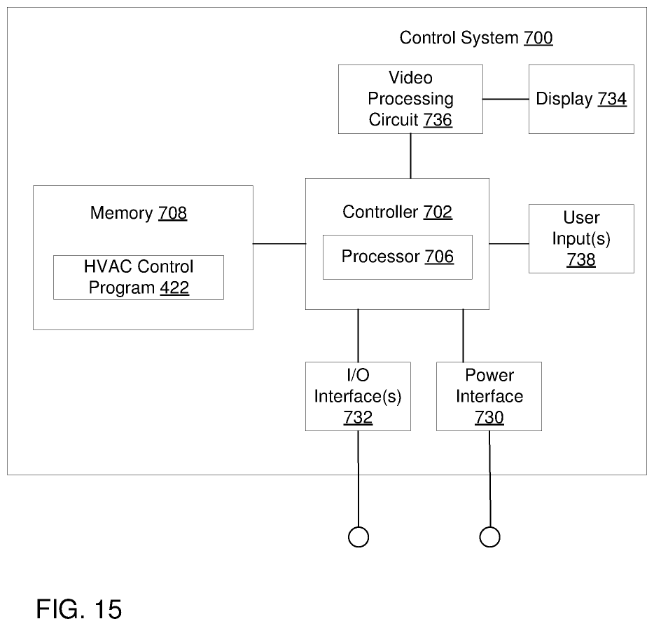

Alternatively, the controller 402 may be communicatively coupled to a separate controller of the HVAC system for controlling the components of the HVAC system. The separate controller may be configured to carry out overall control of the components of the HVAC system, and the separate controller may include a processor (e.g., CPU) for executing code stored in a memory (see e.g., FIG. 15, controller 702). The components of the HVAC system may be controlled by the separate controller based at least in part on values/signals received from controller 402. The controllers may communicate via ORI and/or BACnet communication protocol. In still other embodiments, the one or more signals from the one or more ambient environmental condition sensors may be input directly to the controller for the HVAC system.

In some embodiments, the controller 402 may be communicatively coupled to the air purification system 530. The controller 402 may control the operation of the air purification system based at least in part on the signal of the one or more ambient environmental condition sensors. This control can be accomplished by the controller 402 executing the air purification control program 424 stored in the memory 408 for controlling overall operation of the air purification system 530. In an example, the controller controls the activation, deactivation, and/or setting of the reactive oxygen species (ROS) generator 532 based on the measured ROS amount relative to the set ROS amount. In another example, the controller controls the activation, deactivation, and/or setting of the air purifier 534 (e.g., UV light air purifier) based on the measured particle count relative to the set threshold particle count. In another example, the controller will issue a warning or notification to service the air purification system based on the measured particle count.

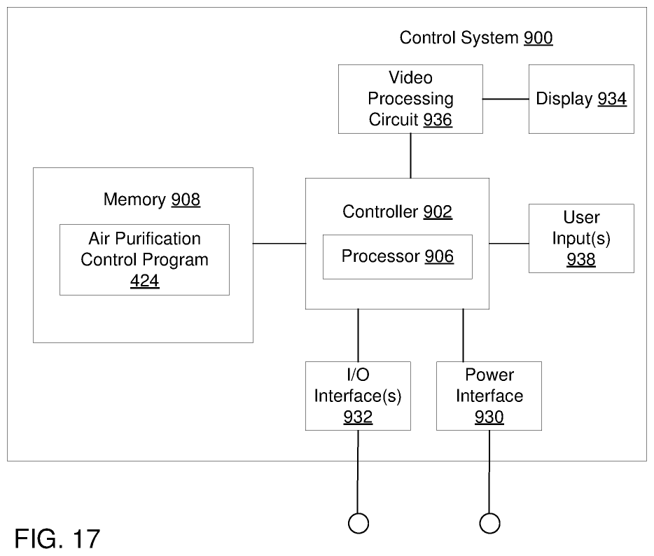

Alternatively, the controller 402 may be communicatively coupled to a separate controller of the air purification system for controlling the components of the air purification system. The separate controller may be configured to carry out overall control of the components of the air purification system, and the separate controller may include a processor (e.g., CPU) for executing code stored in a memory (see e.g., FIG. 17, controller 902). The components of the air purification system may be controlled by the separate controller based at least in part on values/signals received from controller 402. The controllers may communicate via ORI and/or BACnet communication protocol. In still other embodiments, the one or more signals from the one or more ambient environmental condition sensors may be input directly to the controller for the air purification system.

It will be appreciated that in some embodiments the air purification system may be included as part of the HVAC system. In such embodiments, the above-mentioned control associated with the air purification system may be performed as part of the HVAC system control.

In some embodiments, the controller 402 may be communicatively coupled to a patient warming/cooling system 520. The controller 402 may control the operation of the patient warming/cooling system based at least in part on the signal of the one or more ambient environmental condition sensors. This control can be accomplished by the controller 402 executing the patient warming/cooling control program 426 stored in the memory 408 for controlling overall operation of the patient warming/cooling system 520. The controller may be coupled to one or more of the over-body blanket 522, underbody blanket 524, underbody pad 526, and headrest 528. In some embodiments, one or more of the over-body blanket 522, underbody blanket 524, underbody pad 526, and headrest 528 include one or more heating elements (e.g., resistive heating components) that may be controlled by supply of power from the controller. In some embodiments, one or more of the over-body blanket 522, underbody blanket 524, underbody pad 526, and headrest 528 include one or more cooling elements (e.g., vents, water lines, refrigerant lines) that can be controlled by the controller. In an example, the controller controls the supply of power to activate, deactivate, and/or set the temperature setpoint or operation state of the over-body blanket 522, underbody blanket 524, underbody pad 526, and/or headrest 528.

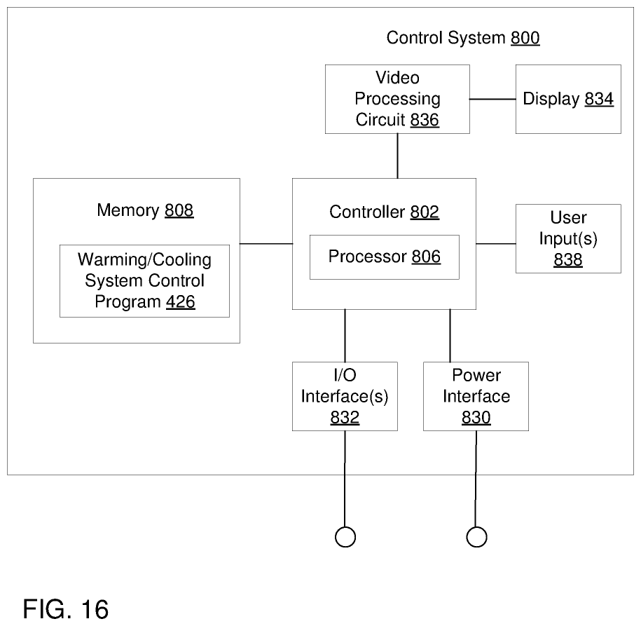

Alternatively, the controller 402 may be communicatively coupled to a separate controller of the patient warming/cooling system for controlling the components of the patient warming/cooling system. The separate controller may be configured to carry out overall control of the components of the patient warming/cooling system, and the separate controller may include a processor (e.g., CPU) for executing code stored in a memory (see e.g., FIG. 16, controller 802). The components of the patient warming/cooling system may be controlled by the separate controller based at least in part on values/signals received from controller 402. The controllers may communicate via ORI and/or BACnet communication protocol. In still other embodiments, the one or more signals from the one or more ambient environmental condition sensors may be input directly to the controller for the patient warming/cooling system.

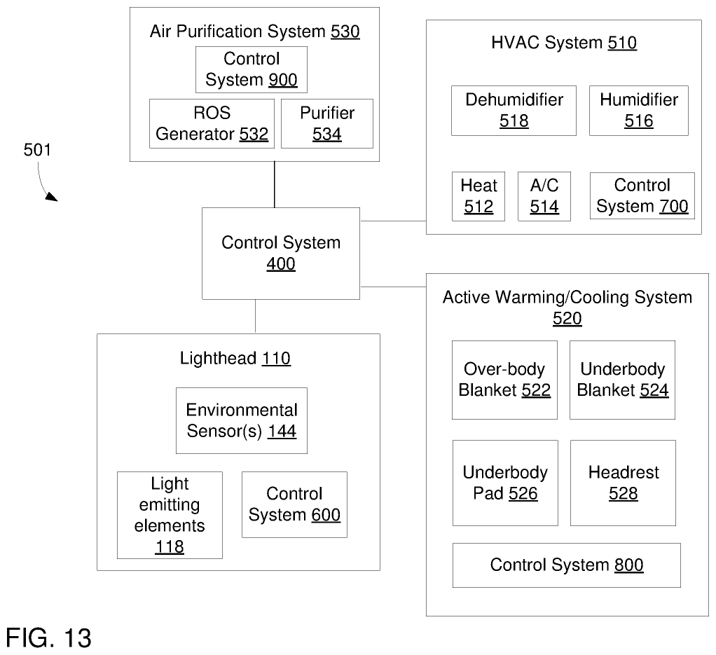

FIG. 13 shows another exemplary arrangement of an environmental condition control system 501. The system differs from that shown in FIG. 12 in that, in addition to the control system 400, additional controllers 602, 702, 802, 902 are respectively associated with the lighthead, HVAC system 510, active warming/cooling system 520, and air purification system 530. In this embodiment, one or more of the lighthead control program 420, HVAC control program 422, air purification control program 424, and patient warming/cooling control program 426 may be omitted from the memory 408 of the control system 400. These programs may instead be respectively included in the memory of the control system 600, 700, 800, 900 associated with the lighthead, HVAC system 510, active warming/cooling system 520, and air purification system 530.

FIG. 14 shows an exemplary control system 600 associated with a lighthead 110. The controller 602 is configured to carry out overall control of the functions and operations of the control system 600. The controller 602 may include a processor 606, such as a central processing unit (CPU), microcontroller, or microprocessor. The processor 606 executes code stored in a memory (not shown) within the controller 602 and/or in a separate memory, such as the memory 608, in order to carry out operation of the lighthead. Lighthead control program 420 is stored in the memory 608. Operating power may be received from an external power source via a power interface 630. One or more input/output (I/O) interface(s) 632 may connect the controller 602 to the controller 402 of control system 400. The control system 600 may include a display 634 and a video processing circuit 636. The control system 600 may include one or more user inputs 638 for receiving user input for controlling operation of the control system 600. The configuration and features of the controller 602, processor 606, memory 608, lighthead control program 422, power interface 430, I/O interface 632, display 634, video processing circuit 636, user inputs 638 may respectively correspond to the configuration and features of the controller 402, processor 406, memory 408, lighthead control program 422, power interface 430, I/O interface 432, display 434, video processing circuit 436, and user inputs 638. The details thereof will not be repeated for sake of brevity.

FIG. 15 shows an exemplary control system 700 associated with HVAC system 510. The controller 702 is configured to carry out overall control of the functions and operations of the control system 700. The controller 702 may include a processor 706, such as a central processing unit (CPU), microcontroller, or microprocessor. The processor 706 executes code stored in a memory (not shown) within the controller 702 and/or in a separate memory, such as the memory 708, in order to carry out operation of the HVAC system. HVAC control program 422 is stored in the memory 708. Operating power may be received from an external power source via a power interface 730. One or more input/output (I/O) interface(s) 732 may connect the controller 702 to the controller 402 of control system 400 and/or to the one or more ambient environmental condition sensors. The control system 700 may include a display 734 and a video processing circuit 736. The control system 700 may include one or more user inputs 738 for receiving user input for controlling operation of the control system 700. The configuration and features of the controller 702, processor 706, memory 708, HVAC control program 422, power interface 730, I/O interface 732, display 734, video processing circuit 736, user inputs 738 may respectively correspond to the configuration and features of the controller 402, processor 406, memory 408, HVAC control program 422, power interface 430, I/O interface 432, display 434, video processing circuit 436, and user inputs 638. The details thereof will not be repeated for sake of brevity.

FIG. 16 shows an exemplary control system 800 associated with patient warming/cooling system 520. The controller 802 is configured to carry out overall control of the functions and operations of the control system 800. The controller 802 may include a processor 806, such as a central processing unit (CPU), microcontroller, or microprocessor. The processor 806 executes code stored in a memory (not shown) within the controller 802 and/or in a separate memory, such as the memory 808, in order to carry out operation of the patient warming/cooling system. Patient warming/cooling control program 426 is stored in the memory 808. Operating power may be received from an external power source via a power interface 830. One or more input/output (I/O) interface(s) 832 may connect the controller 802 to the controller 402 of control system 400 and/or to the one or more ambient environmental condition sensors. The control system 800 may include a display 834 and a video processing circuit 836. The control system 800 may include one or more user inputs 838 for receiving user input for controlling operation of the control system 800. The configuration and features of the controller 802, processor 806, memory 808, patient warming/cooling control program 426, power interface 830, I/O interface 832, display 834, video processing circuit 836, user inputs 838 may respectively correspond to the configuration and features of the controller 402, processor 406, memory 408, patient warming/cooling control program 426, power interface 430, I/O interface 432, display 434, video processing circuit 436, and user inputs 638. The details thereof will not be repeated for sake of brevity.

FIG. 17 shows an exemplary control system 900 associated with air purification system 530. The controller 902 is configured to carry out overall control of the functions and operations of the control system 900. The controller 902 may include a processor 906, such as a central processing unit (CPU), microcontroller, or microprocessor. The processor 906 executes code stored in a memory (not shown) within the controller 902 and/or in a separate memory, such as the memory 908, in order to carry out operation of the air purification system. Air purification control program 424 is stored in the memory 908. Operating power may be received from an external power source via a power interface 930. One or more input/output (I/O) interface(s) 932 may connect the controller 902 to the controller 902 of control system 900 and/or to the one or more ambient environmental condition sensors. The control system 900 may include a display 934 and a video processing circuit 936. The control system 900 may include one or more user inputs 938 for receiving user input for controlling operation of the control system 900. The configuration and features of the controller 902, processor 906, memory 908, air purification control program 424, power interface 930, I/O interface 932, display 934, video processing circuit 936, user inputs 938 may respectively correspond to the configuration and features of the controller 402, processor 406, memory 408, air purification control program 424, power interface 430, I/O interface 432, display 434, video processing circuit 436, and user inputs 638. The details thereof will not be repeated for sake of brevity.

In the exemplary system shown in FIG. 13, the control system 400 can receive a signal from the environmental sensor, process the signal, and can output one or more processed signals/values that are used as an input to the controller 602 of the lighthead, the controller 702 of the HVAC system, the controller 802 of the patient warming/cooling system 520, and/or the controller 902 of the air purification system 530 to control one or more environmental conditions at the region of interest.

In an example, the controller 602 controls the radiant power of the light emitting elements 118 based at least in part on the signal/value received from the controller 402. In another example, the controller 602 controls the position of the lens to adjust the light output distribution of the light emitting elements based at least in part on the signal/value received from the controller 402.

In an example, the controller 702 controls the activation, deactivation, and/or setting of one or more of the blower, heater, compressor, condenser, and vents respectively associated with the furnace 512 and A/C 514 systems of the HVAC system based on the measured ambient temperature relative to the temperature setpoint. In another example, the controller 702 controls the activation, deactivation, and/or setting of a dehumidifier 518 or humidifier 516 based on the measured relative humidity relative to the set relative humidity.

In an example, the controller 802 controls the supply of power to activate, deactivate, and/or set the temperature setpoint or operation state of the over-body blanket 522, underbody blanket 524, underbody pad 526, and/or headrest 528.

In an example, the controller 902 controls the activation, deactivation, and/or setting of the reactive oxygen species generator based on the measured ROS amount relative to the set ROS amount. In another example, the controller 902 controls the activation, deactivation, and/or setting of the air purifier (e.g., UV light air purifier) based on the measured particle count relative to the set threshold particle count. In another example, the controller 902 will issue a warning or notification to service the air purification system based on the measured particle count.

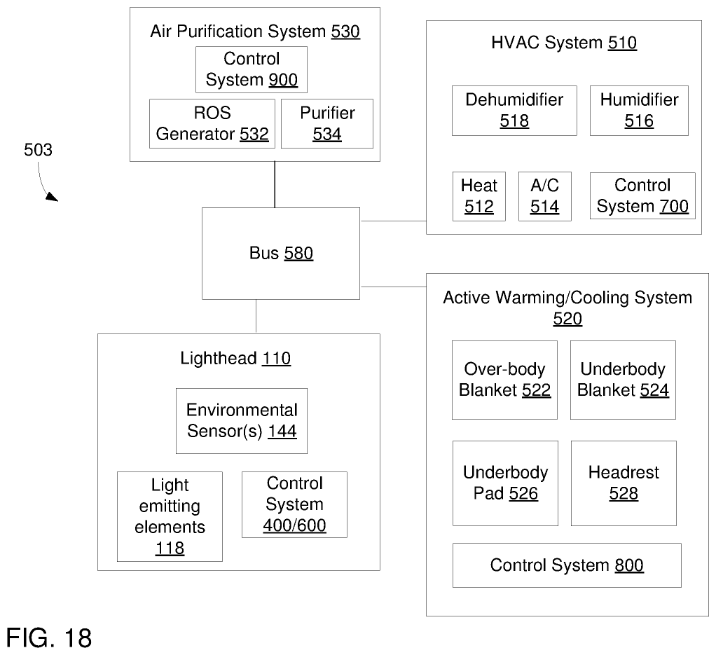

It will be appreciated that in other embodiments, the controllers of the system can be differently arranged and configured. For example, in some embodiments, the control systems 400, 600, 700, 800, 900 may respectively receive one or more signals from the one or more environmental sensors and may process the one or more signals and control the system based thereon. The control system 400, 600, 700, 800, 900 may respectively include one or more of temperature sensing program 410, humidity sensing program 412, barometric pressure sensing program 414, particle count sensing program 416, and ROS sensing program 418, depending on what signal is received by the controller. FIG. 18 shows another exemplary arrangement of an environmental condition control system 503 in which the lighthead 110 includes control system 400/600, and HVAC system 510, patient warming/cooling system 520, and air purification system 530 are communicatively coupled to the lighthead via bus 580. The one or more signals from the one or more environmental sensors may be input directly to each control system 400/600, 700, 800, 900.

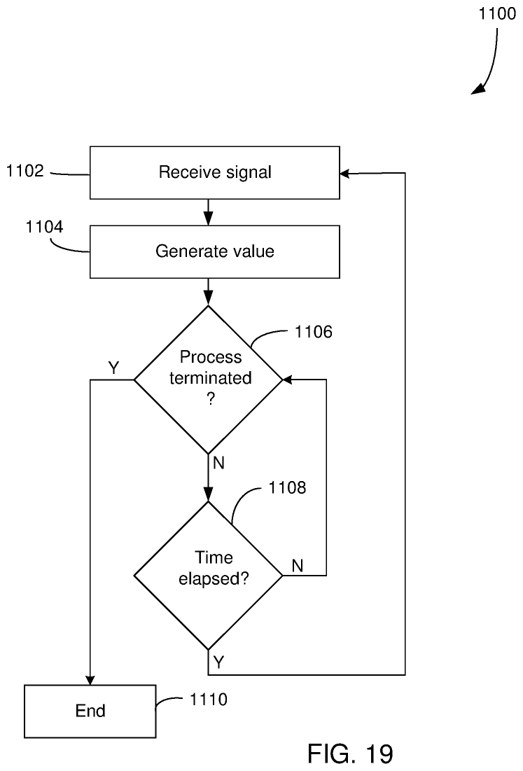

FIG. 19 is a flowchart showing an exemplary process 1100 for determining a value of an environmental condition. In some embodiments, the process described in FIG. 19 is performed by the controller 402, 602, 702, 802, 902 executing one of the temperature sensing program 410, humidity sensing program 412, barometric pressure sensing program 414, particle count sensing program 416, or ROS sensing program 418. In embodiment in which more than one type of ambient environmental condition sensor is included, the process 1100 may be performed for each sensor. It will also be appreciated that in some embodiments, more than one of a particular type of ambient environmental condition sensor is included. For example, more than one lighthead having ambient environmental condition sensors is present. In such embodiments, control may be based off of signals input from all of the sensor(s) of that particular type. For example, the temperature values can be averaged, or comparison to a temperature setpoint or range can be performed for each temperature value.

At step 1102, the signal is received from the ambient environmental condition sensor. In some embodiments, the signal is an electrical signal indicative of the environmental condition.

At step 1104 a value is generated based on the signal that is representative of the environmental condition. In some embodiments, one or more values, reference values, references signals, and/or information may be stored in the memory for use in generating the value. The signal may be compared against a reference signal, and the generated value may be a value that is associated with to the reference signal corresponding to the signal received form the ambient environmental condition sensor.

At step 1106, it is determined whether the process is terminated. The process may be, for example, a detecting process during an on state of the system. If yes, the process ends at step 1110. If no, the process proceeds to step 1108 where it is determined whether a predetermined amount of time has lapsed. The predetermined amount of time may be any suitable amount of time. Depending on the ambient environmental condition sensor, the predetermined amount of time may be, for example, 1 second, 10 seconds, 30 seconds, 1 minute, or a different amount of time.

If the predetermined amount time has not elapsed (no), the process reverts to step 1106. If the predetermined amount time has elapsed (yes), the process returns to step 1102.

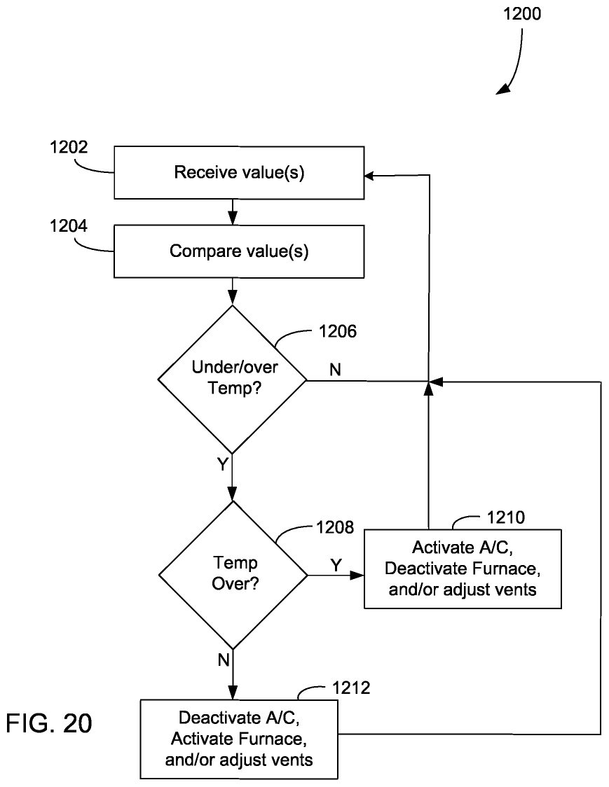

FIGS. 20-25 are exemplary processes for controlling an environmental condition based at least in part on the generated value.

FIG. 20 is a flowchart showing an exemplary process 1200 for controlling temperature at the region of interest based at least in part on the temperature measured by the temperature sensor 152. In some embodiments, the process described in FIG. 20 is performed by the controller 402, 702 executing the HVAC control program 420.

At step 1202, the value representative of the environmental condition is received. In some embodiments, a single value is received. In other embodiments, such as those in which more than one value is present, two or more values are received.

At step 1204, the received value(s) is compared against a set point or set temperature range. In some embodiments, the received value is compared as an individual number. In embodiments in which more than one sensor is included as a part of the system, each received value may be individually compared. In other embodiments, the received value is compared as part of a group of two or more numbers. For example, the received value can be compared as part of a rolling average of values over a predetermined time period. For example, an average of the values received over the past minute may be used. This can be performed for the value received from each sensor. In another example in which there is more than one sensor, an average of the values from the sensors for a given point in time can be used for the comparison.

At step 1206, it is determined whether the received value(s) is outside of the setpoint or the predetermined range. If no, the process reverts back to step 1202. If yes, the process proceeds to step 1208 to control the temperature. If the temperature is over (step 1208, yes), the process proceeds to step 1210 where one or more of the A/C activated, the furnace is deactivated, and the vents of the HVAC system are adjusted. If the temperature is under (step 1208, no), the process proceeds to step 1212 where one or more of the A/C deactivated, the furnace is activated, and the vents of the HVAC system are adjusted. The process then reverts to step 1202 where new values are received.

It will be appreciated that in some embodiments, the process 1200 may include a time delay from when the received value(s) is determined to be outside of the setpoint or the predetermined range (steps 1206, 1208) until when one or more components of the environmental condition control system are controlled (steps 1210, 1212). For example, upon each of determining at step 1208 that the temperature is over (step 1208, yes) or under (step 1208, no), it may be subsequently determined whether a predetermined amount of time has elapsed. This predetermined amount of time may be any suitable amount of time (e.g., 10 seconds, 30 seconds, 1 minute, etc.) and may be compared to an amount of time that the instance of the over or under condition has been occurring. For example, when consecutive over or when consecutive under values are read, the total amount of time over which the consecutive readings are read may be compared to the predetermined amount of time. If the total amount of time is less than the predetermined amount of time, the process may revert to step 1202 without controlling the one or more components of the environmental condition control system. If the total amount of time is equal to or greater than the predetermined amount of time, the process may proceed to step 1210 or 1212 to control the one or more components of the environmental condition control system. The total amount of time may reset once the received value(s) is no longer over/under. This approach may allow for minor incidental fluctuations in the environmental conditions.

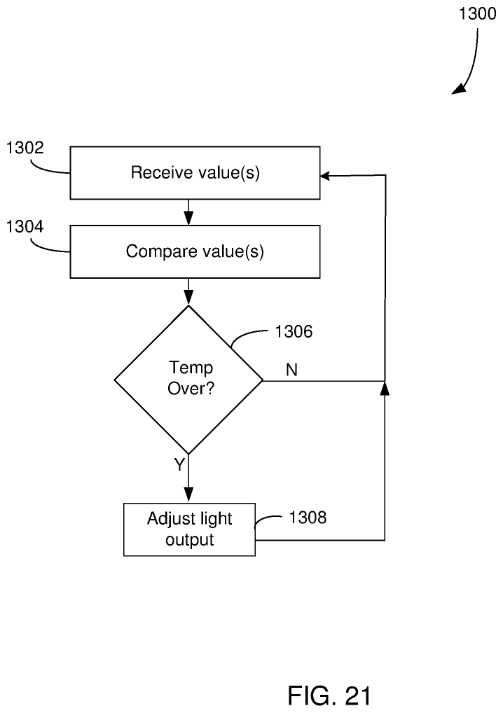

FIG. 21 is a flowchart showing an exemplary process 1300 for controlling temperature at the region of interest based at least in part on the temperature measured by the temperature sensor 152. In some embodiments, the process described in FIG. 21 is performed by the controller 402, 602 executing the lighthead control program 420.

At step 1302, the value representative of the environmental condition is received. In some embodiments, a single value is received. In other embodiments, such as those in which more than one value is present, two or more values are received.

At step 1304, the received value(s) is compared against a set point or set temperature range. In some embodiments, the received value is compared as an individual number. In embodiments in which more than one sensor is included as a part of the system, each received value may be individually compared. In other embodiments, the received value is compared as part of a group of two or more numbers. For example, the received value can be compared as part of a rolling average of values over a predetermined time period. For example, an average of the values received over the past minute may be used. This can be performed for the value received from each sensor. In another example in which there is more than one sensor, an average of the values from the sensors for a given point in time can be used for the comparison.

At step 1306, it is determined whether the received value(s) is over the setpoint or the predetermined range. If no, the process reverts back to step 1202. If yes, the process proceeds to step 1208 where the light output from the lighthead is adjusted. Adjustment may include one or more light output intensity and light output distribution. The process then reverts to step 1202 where new values are received.

It will be appreciated that in some embodiments, the process 1300 may include a time delay from when the received value(s) is determined to be over the setpoint or the predetermined range (step 1306) until when the light output is adjusted (step 1308). For example, upon each of determining at step 1306 that the temperature is over (step 1306, yes), it may be subsequently determined whether a predetermined amount of time has elapsed. This predetermined amount of time may be any suitable amount of time (e.g., 10 seconds, 30 seconds, 1 minute, etc.) and may be compared to an amount of time that the instance of the over condition has been occurring. For example, when consecutive over values are read, the total amount of time over which the consecutive readings are read may be compared to the predetermined amount of time. If the total amount of time is less than the predetermined amount of time, the process may revert to step 1302 without adjusting the light output. If the total amount of time is equal to or greater than the predetermined amount of time, the process may proceed to step 1308 to adjust the light output. The total amount of time may reset once the received value(s) is no longer over. This approach may allow for minor incidental fluctuations in the environmental conditions.

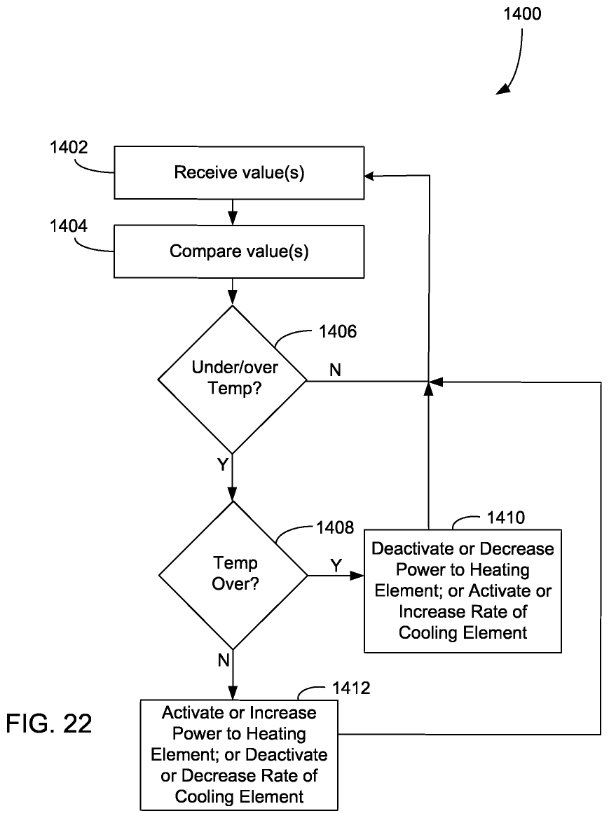

FIG. 22 is a flowchart showing an exemplary process 1400 for controlling temperature at the region of interest based at least in part on the temperature measured by the temperature sensor 152. In some embodiments, the process described in FIG. 22 is performed by the controller 402, 802 executing the patient warming/cooling control program 420.

At step 1402, the value representative of the environmental condition is received. In some embodiments, a single value is received. In other embodiments, such as those in which more than one value is present, two or more values are received.

At step 1404, the received value(s) is compared against a set point or set temperature range. In some embodiments, the received value is compared as an individual number. In embodiments in which more than one sensor is included as a part of the system, each received value may be individually compared. In other embodiments, the received value is compared as part of a group of two or more numbers. For example, the received value can be compared as part of a rolling average of values over a predetermined time period. For example, an average of the values received over the past minute may be used. This can be performed for the value received from each sensor. In another example in which there is more than one sensor, an average of the values from the sensors for a given point in time can be used for the comparison.

At step 1406, it is determined whether the received value(s) is outside of the setpoint or the predetermined range. If no, the process reverts back to step 1402. If yes, the process proceeds to step 1408 to control the temperature. If the temperature is over (step 1408, yes), the process proceeds to step 1410 where one or more of the heating element of one or more of the devices is deactivated, power to one or more of the heating elements is decreased, one or more cooling elements of the device is activated, and the cooling rate of the cooling elements is increased. If the temperature is under (step 1408, no), the process proceeds to step 1412 where one or more of the heating element of one or more of the devices is activated, power to one or more of the heating elements is increased, one or more cooling elements of the device is deactivated, and the cooling rate of the cooling elements is decreased. The process then reverts to step 1402 where new values are received.

It will be appreciated that in some embodiments, the process 1400 may include a time delay from when the received value(s) is determined to be outside of the setpoint or the predetermined range (steps 1406, 1408) until when one or more components of the environmental condition control system are controlled (steps 1410, 1412). For example, upon each of determining at step 1408 that the temperature is over (step 1408, yes) or under (step 1408, no), it may be subsequently determined whether a predetermined amount of time has elapsed. This predetermined amount of time may be any suitable amount of time (e.g., 10 seconds, 30 seconds, 1 minute, etc.) and may be compared to an amount of time that the instance of the over or under condition has been occurring. For example, when consecutive over or when consecutive under values are read, the total amount of time over which the consecutive readings are read may be compared to the predetermined amount of time. If the total amount of time is less than the predetermined amount of time, the process may revert to step 1402 without controlling the one or more components of the environmental condition control system. If the total amount of time is equal to or greater than the predetermined amount of time, the process may proceed to step 1410 or 1412 to control the one or more components of the environmental condition control system. The total amount of time may reset once the received value(s) is no longer over/under. This approach may allow for minor incidental fluctuations in the environmental conditions.

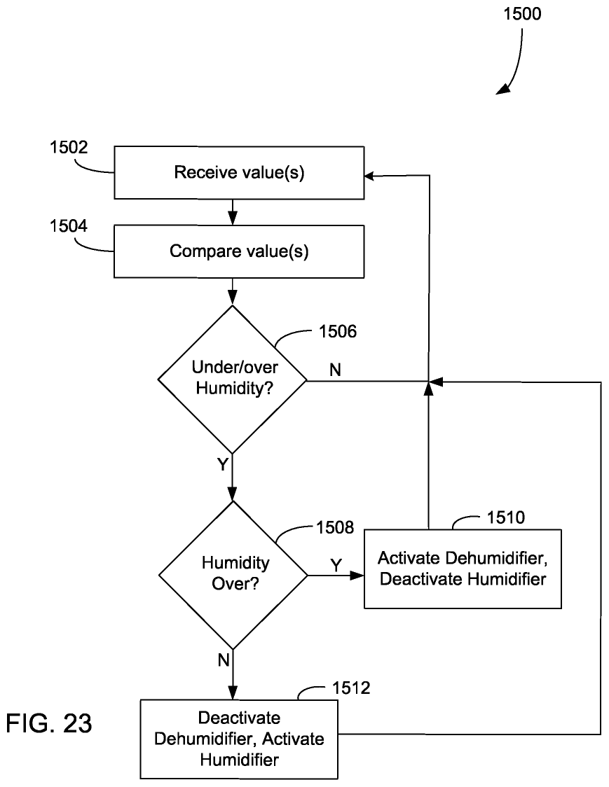

FIG. 23 is a flowchart showing an exemplary process 1500 for controlling relative humidity at the region of interest based at least in part on the relative humidity measured by the humidity sensor 154. In some embodiments, the process described in FIG. 23 is performed by the controller 402, 702 executing the HVAC control program 420.

At step 1502, the value representative of the environmental condition is received. In some embodiments, a single value is received. In other embodiments, such as those in which more than one value is present, two or more values are received.

At step 1504, the received value(s) is compared against a set point or set relative humidity range. In some embodiments, the received value is compared as an individual number. In embodiments in which more than one sensor is included as a part of the system, each received value may be individually compared. In other embodiments, the received value is compared as part of a group of two or more numbers. For example, the received value can be compared as part of a rolling average of values over a predetermined time period. For example, an average of the values received over the past minute may be used. This can be performed for the value received from each sensor. In another example in which there is more than one sensor, an average of the values from the sensors for a given point in time can be used for the comparison.

At step 1506, it is determined whether the received value(s) is outside of the setpoint or the predetermined range. If no, the process reverts back to step 1502. If yes, the process proceeds to step 1508 to control the relative humidity. If the relative humidity is over (step 1508, yes), the process proceeds to step 1510 where one or more of the dehumidifier is activated and the humidifier is deactivated. If the relative humidity is under (step 1508, no), the process proceeds to step 1512 where one or more of the dehumidifier is deactivated and the humidifier is activated. The process then reverts to step 1502 where new values are received.

It will be appreciated that in some embodiments, the process 1500 may include a time delay from when the received value(s) is determined to be outside of the setpoint or the predetermined range (steps 1506, 1508) until when one or more components of the environmental condition control system are controlled (steps 1510, 1512). For example, upon each of determining at step 1508 that the humidity is over (step 1508, yes) or under (step 1508, no), it may be subsequently determined whether a predetermined amount of time has elapsed. This predetermined amount of time may be any suitable amount of time (e.g., 10 seconds, 30 seconds, 1 minute, etc.) and may be compared to an amount of time that the instance of the over or under condition has been occurring. For example, when consecutive over or when consecutive under values are read, the total amount of time over which the consecutive readings are read may be compared to the predetermined amount of time. If the total amount of time is less than the predetermined amount of time, the process may revert to step 1502 without controlling the one or more components of the environmental condition control system. If the total amount of time is equal to or greater than the predetermined amount of time, the process may proceed to step 1510 or 1512 to control the one or more components of the environmental condition control system. The total amount of time may reset once the received value(s) is no longer over/under. This approach may allow for minor incidental fluctuations in the environmental conditions.

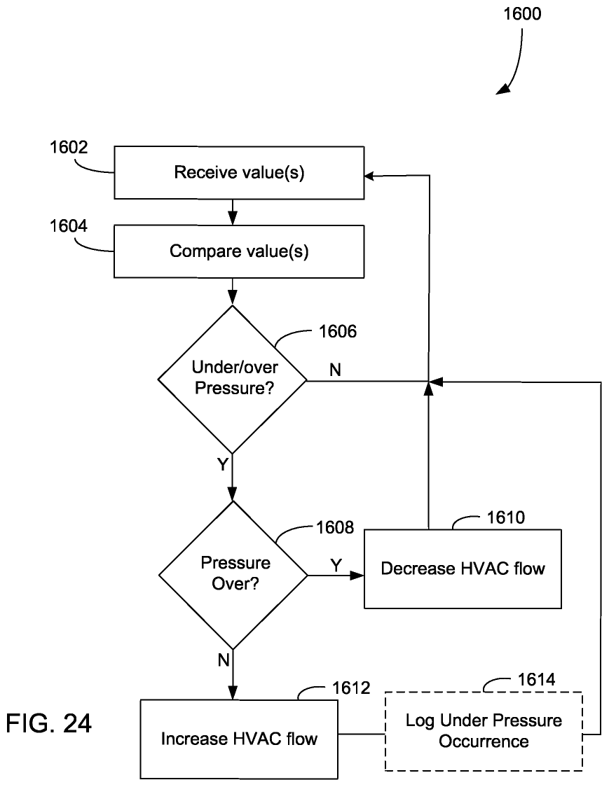

FIG. 24 is a flowchart showing an exemplary process 1600 for monitoring controlling atmospheric pressure at the region of interest based at least in part on the pressure measured by the pressure sensor 158. In some embodiments, the process described in FIG. 24 is performed by the controller 402, 702 executing the HVAC control program 420.

At step 1602, the value representative of the environmental condition is received. In some embodiments, a single value is received. In other embodiments, such as those in which more than one value is present, two or more values are received.

At step 1604, the received value(s) is compared against a set point or set atmospheric pressure range. In some embodiments, the received value is compared as an individual number. In embodiments in which more than one sensor is included as a part of the system, each received value may be individually compared. In other embodiments, the received value is compared as part of a group of two or more numbers. For example, the received value can be compared as part of a rolling average of values over a predetermined time period. For example, an average of the values received over the past minute may be used. This can be performed for the value received from each sensor. In another example in which there is more than one sensor, an average of the values from the sensors for a given point in time can be used for the comparison.

At step 1606, it is determined whether the received value(s) is outside of the setpoint or the predetermined range. If no, the process reverts back to step 1602. If yes, the process proceeds to step 1608 to control the atmospheric pressure. If the atmospheric pressure is over (step 1608, yes), the process proceeds to step 1610 where HVAC flow is decreased using one or more of the blower motor and vents. If the atmospheric pressure is under (step 1608, no), the process proceeds to step 1612 where HVAC flow is increased using one or more of the blower motor and vents. Optionally at step 1614, a log of the under-pressure occurrence is recorded and stored. The log may be stored in the memory of the control system. The process then reverts to step 1602 where new values are received.

It will be appreciated that in some embodiments, the process 1600 may include a time delay from when the received value(s) is determined to be outside of the setpoint or the predetermined range (steps 1606, 1608) until when one or more components of the environmental condition control system are controlled (steps 1610, 1612). For example, upon each of determining at step 1608 that the pressure is over (step 1608, yes) or under (step 1608, no), it may be subsequently determined whether a predetermined amount of time has elapsed. This predetermined amount of time may be any suitable amount of time (e.g., 10 seconds, 30 seconds, 1 minute, etc.) and may be compared to an amount of time that the instance of the over or under condition has been occurring. For example, when consecutive over or when consecutive under values are read, the total amount of time over which the consecutive readings are read may be compared to the predetermined amount of time. If the total amount of time is less than the predetermined amount of time, the process may revert to step 1602 without controlling the one or more components of the environmental condition control system. If the total amount of time is equal to or greater than the predetermined amount of time, the process may proceed to step 1610 or 1612 to control the one or more components of the environmental condition control system. The total amount of time may reset once the received value(s) is no longer over/under. This approach may allow for minor incidental fluctuations in the environmental conditions.

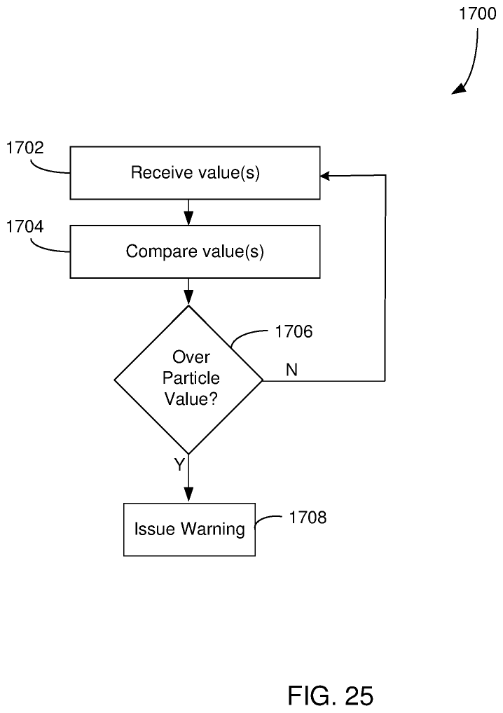

FIG. 25 is a flowchart showing an exemplary process 1700 for monitoring particle concentration at the region of interest based at least in part on the particle count measured by the particle counter 162. In some embodiments, the process described in FIG. 25 is performed by the controller 402, 902 executing the air purification control program 420.

At step 1702, the value representative of the environmental condition is received. In some embodiments, a single value is received. In other embodiments, such as those in which more than one value is present, two or more values are received.

At step 1704, the received value(s) is compared against a set point or set particle range. In some embodiments, the received value is compared as an individual number. In embodiments in which more than one sensor is included as a part of the system, each received value may be individually compared. In other embodiments, the received value is compared as part of a group of two or more numbers. For example, the received value can be compared as part of a rolling average of values over a predetermined time period. For example, an average of the values received over the past minute may be used. This can be performed for the value received from each sensor. In another example in which there is more than one sensor, an average of the values from the sensors for a given point in time can be used for the comparison.

At step 1706, it is determined whether the received value(s) is outside of the setpoint or the predetermined range. If no, the process reverts back to step 1702. If yes, the process proceeds to step 1708 where a warning is issued. The warning may in some embodiments be displayed on the display of the control system. In some embodiments, the warning may also prompt a request for service.

It will be appreciated that in some embodiments, the process 1700 may include a time delay from when the received value(s) is determined to be over the setpoint or the predetermined range (step 1706) until when the light output is adjusted (step 1708). For example, upon each of determining at step 1706 that the particle value is over (step 1706, yes), it may be subsequently determined whether a predetermined amount of time has elapsed. This predetermined amount of time may be any suitable amount of time (e.g., 10 seconds, 30 seconds, 1 minute, etc.) and may be compared to an amount of time that the instance of the over condition has been occurring. For example, when consecutive over values are read, the total amount of time over which the consecutive readings are read may be compared to the predetermined amount of time. If the total amount of time is less than the predetermined amount of time, the process may revert to step 1702 without adjusting the light output. If the total amount of time is equal to or greater than the predetermined amount of time, the process may proceed to step 1708 to adjust the light output. The total amount of time may reset once the received value(s) is no longer over. This approach may allow for minor incidental fluctuations in the environmental conditions.

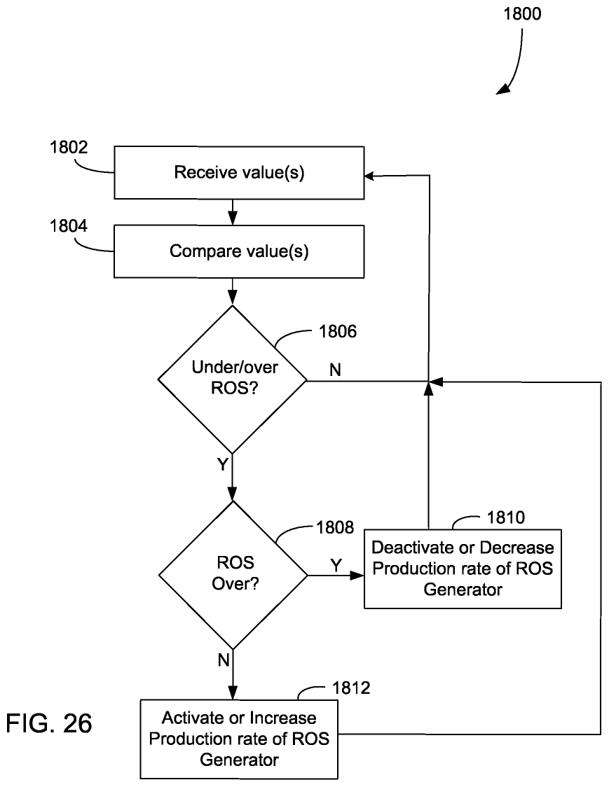

FIG. 26 is a flowchart showing an exemplary process 1800 for controlling ROS at the region of interest based at least in part on the concentration of ROS measured by the ROS sensor 164. In some embodiments, the process described in FIG. 26 is performed by the controller 402, 902 executing the air purification control program 424.

At step 1802, the value representative of the environmental condition is received. In some embodiments, a single value is received. In other embodiments, such as those in which more than one value is present, two or more values are received.

At step 1804, the received value(s) is compared against a set point or set ROS range. In some embodiments, the received value is compared as an individual number. In embodiments in which more than one sensor is included as a part of the system, each received value may be individually compared. In other embodiments, the received value is compared as part of a group of two or more numbers. For example, the received value can be compared as part of a rolling average of values over a predetermined time period. For example, an average of the values received over the past minute may be used. This can be performed for the value received from each sensor. In another example in which there is more than one sensor, an average of the values from the sensors for a given point in time can be used for the comparison.

At step 1806, it is determined whether the received value(s) is outside of the setpoint or the predetermined range. If no, the process reverts back to step 1802. If yes, the process proceeds to step 1808 to control the ROS concentration. If the ROS concentration is over (step 1808, yes), the process proceeds to step 1810 where one or more of the ROS generator is deactivated or the production rate of the ROS generator is decreased. If the ROS concentration is under (step 1808, no), the process proceeds to step 1812 where one or more of the ROS generator is activated or the production rate of the ROS generator is increased. The process then reverts to step 1802 where new values are received.

It will be appreciated that in some embodiments, the process 1800 may include a time delay from when the received value(s) is determined to be outside of the setpoint or the predetermined range (steps 1806, 1808) until when one or more components of the environmental condition control system are controlled (steps 1810, 1812). For example, upon each of determining at step 1808 that the ROS concentration is over (step 1808, yes) or under (step 1808, no), it may be subsequently determined whether a predetermined amount of time has elapsed. This predetermined amount of time may be any suitable amount of time (e.g., 10 seconds, 30 seconds, 1 minute, etc.) and may be compared to an amount of time that the instance of the over or under condition has been occurring. For example, when consecutive over or when consecutive under values are read, the total amount of time over which the consecutive readings are read may be compared to the predetermined amount of time. If the total amount of time is less than the predetermined amount of time, the process may revert to step 1802 without controlling the one or more components of the environmental condition control system. If the total amount of time is equal to or greater than the predetermined amount of time, the process may proceed to step 1810 or 1812 to control the one or more components of the environmental condition control system. The total amount of time may reset once the read value is no longer over/under. This approach may allow for minor incidental fluctuations in the environmental conditions.

In another embodiment a PID loop control can be used to control the generator to create the desired ROS at the sensor.

Although the invention has been shown and described with respect to certain preferred embodiments, it is understood that equivalents and modifications will occur to others skilled in the art upon the reading and understanding of the specification and the attached drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application. The present invention includes all such equivalents and modifications and is limited only by the scope of the following claims.

1. A system, comprising:
a surgical lighthead comprising a lighthead housing including a plurality of light emitting elements therein that are arranged to emit light toward to a region of interest, and a handle attached to the lighthead housing;
a sensor integrated with the lighthead and configured to measure an ambient environmental condition at the lighthead; and
a controller operatively coupled to the sensor and configured to detect an ambient environmental condition based at least in part on the measured ambient environmental condition by the sensor.

2. The system of claim 1, wherein the sensor is one of a thermistor, a thermocouple, a thermometer or an infrared temperature sensor and the ambient environmental condition is temperature.

3.-5. (canceled)

6. The system of claim 1, wherein the sensor is a hygrometer and the ambient environmental condition is humidity.

7. The system of claim 1, wherein the sensor is a barometer and the ambient environmental condition is atmospheric pressure.

8. The system of claim 1, wherein the lighthead comprises one or more baffles configured to direct airflow to the sensor.

9. The system of claim 1, wherein the sensor is a particle counter and the ambient environmental condition is concentration of particles in the air.

10. The system of claim 1, wherein the sensor is a reactive oxygen species sensor and the ambient environmental condition is concentration of reactive oxygen species in the air.

11. The system of claim 9, wherein the sensor is located in a flow path in the handle, an air inlet of the flow path is in fluid communication with the sensor, and an air outlet of the flow path is in fluid communication with the sensor.

12. The system of claim 11, wherein the handle comprises an air filter.

13. The system of claim 12, wherein the sensor is integrated with the handle of the lighthead.

14. The system of claim 1, wherein the sensor is housed in the housing of the lighthead.

15. The system of claim 1, wherein the controller is configured to control the ambient environmental condition based at least in part on the measured ambient environmental condition by the sensor.

16. The system of claim 15, wherein the controller is configured to control an HVAC system based at least in part on the ambient environmental condition measured by the sensor.

17. The system of claim 15, wherein the controller is configured to control an air purification system based at least in part on the ambient environmental condition measured by the sensor.

18. The system of claim 15, wherein the controller is configured to control a heated or cooled blanket based at least in part on the ambient environmental condition measured by the sensor.

19. The system of claim 15, wherein the controller is configured to control a heated or cooled underbody pad based at least in part on the ambient environmental condition measured by the sensor.

20. The system of claim 15, wherein the controller is configured to control a heated or cooled headrest based at least in part on the ambient environmental condition measured by the sensor.

21. The system of claim 15, wherein the controller is configured to control light output intensity of the plurality of the light emitting elements based at least in part on the ambient environmental condition measured by the sensor.

22.-32. (canceled)

33. The system of claim 1, wherein:
the sensor is located in a flow path in the handle, an air inlet of the flow path is in fluid communication with the sensor, and an air outlet of the flow path is in fluid communication with the sensor;
the handle comprises an air filter; and
the sensor is integrated with the handle of the lighthead.

34. The system of claim 33, wherein the controller is configured to control the ambient environmental condition based at least in part on the measured ambient environmental condition by the sensor.
